(19) United States
(12) Patent Application Publication
(10) Pub. No.: US 2019/0105264 A1
(43) Pub. Date: Apr. 11, 2019

(54) NON-INVASIVE OCULAR DRUG DELIVERY DEVICES

(71) Applicant: Aciont Inc., Salt Lake City, UT (US)

(72) Inventors: John W. Higuchi, Salt Lake City, UT (US); Kongnara Papangkorn, Sandy, UT (US)

(73) Assignee: Aciont Inc., Salt Lake City, UT (US)

(21) Appl. No.: 15/727,529

(22) Filed: Oct. 6, 2017

(51) Int. Cl. A61K 9/00; A61F 9/00

(52) U.S. Cl. CPC A61K 9/0051; A61B 2017/00893; A61F 9/0017

(57) ABSTRACT

A non-invasive ocular drug delivery device can include a housing adapted to couple to an eye of a subject. An active agent matrix can be coupled to the housing. The active agent matrix can include an electro spun material having a combination of a density, a thickness, and an ocular surface area configured to hold and retain an active agent prior to application of the device to the eye, and deliver an effective dose of an active agent within 30 minutes of application of the device to the eye.

### BACKGROUND

The eyes are subject to numerous adverse health conditions, such as glaucoma, uveitis, age-related macular degeneration, and many other ocular conditions, which can cause impaired vision and eventually lead to blindness. In many instances, ophthalmic drops offer a first line of treatment for ocular disorders. However, eye drops often need to be administered frequently to be effective, which can lead to poor patient compliance and reduced effectiveness of the treatment. Further eye drops often are ineffective at delivering adequate amounts of therapeutic agents into the eye, especially the posterior segment of the eye. Other methods of treating ocular disorders can include painful ocular injections and other invasive procedures, which can have safety risks such as infection, retinal detachment, vitreous hemorrhage, etc.

### BRIEF DESCRIPTION OF THE DRAWINGS

For a fuller understanding of the nature and advantage of the present invention, reference is being made to the following detailed description of preferred embodiments and in connection with the accompanying drawings, in which:

FIG. 1a illustrates a front cross-sectional view of a non-invasive ocular drug delivery device, in accordance with an example embodiment.

FIG. 1b illustrates a bottom view of the non-invasive ocular drug delivery device of FIG. 1a.

FIG. 2a illustrates a front cross-sectional view of a non-invasive ocular drug delivery device, in accordance with an example embodiment.

FIG. 2b illustrates a bottom perspective view of the non-invasive ocular drug delivery device of FIG. 2a.

FIG. 2c illustrates a bottom view of the non-invasive ocular drug delivery device of FIG. 2c.

FIG. 3a illustrates a perspective view of a non-invasive ocular drug delivery device, in accordance with an example embodiment.

FIG. 3b illustrates a side cross-sectional view of the non-invasive ocular drug delivery device of FIG. 3a.

FIG. 3c illustrates a top view of the non-invasive ocular drug delivery device of FIG. 3a.

FIG. 3d illustrates a bottom view of the non-invasive ocular drug delivery device of FIG. 3a.

FIG. 4 illustrates a side cross-sectional view of the device of FIG. 3a attached to an eye, in accordance with an example embodiment.

FIG. 5a illustrates a top view of a loading base, in accordance with an example embodiment.

FIG. 5b illustrates a side cross-sectional view of a loading base with the non-invasive ocular drug delivery device of FIG. 3a coupled thereto, in accordance with an example embodiment.

FIG. 6 is a graph of the drug release profile of a non-invasive ocular drug delivery device, in accordance with an example embodiment.

FIG. 7 is a graph of vitreous scores of various treatment groups tested in an experimental uveitis rabbit model.

FIG. 8a is a magnified image of a posterior section of an untreated eye depicting severe inflammation and damaged photoreceptor layer (arrow).

FIG. 8b is a magnified image of a posterior section of an eye treated with 15% DSP (15 minutes, 4 doses) depicting minimal inflammation and well-preserved tissue structure.

FIG. 9 is a graph (mean±SD, n=6 eyes) of the amount of drug in the eye, application time, and DSP concentration after single administration of drug via a non-invasive ocular drug delivery device.

FIG. 10a is a graph of mean plasma concentration of DSP (solid line) and DEX (dotted line) following single administration of DSP via a non-invasive ocular drug delivery device.

FIG. 10b is a graph of mean plasma concentration of DSP equivalent following single administration of DSP via a non-invasive ocular drug delivery device. The data were calculated from FIG. 10a based on the sum of DSP and DEX in gram equivalent. No standard deviation is given. To reveal all pharmacokinetic data, graph was not plotted in a linear time sale on the x-axis.

### DESCRIPTION OF EMBODIMENTS

Although the following detailed description contains many specifics for the purpose of illustration, a person of ordinary skill in the art will appreciate that many variations and alterations to the following details can be made and are considered to be included herein. Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

As used in this written description, the singular forms “a,” “an” and “the” include express support for plural referents unless the context clearly dictates otherwise. Thus, for example, reference to “a polymer” can include a plurality of such polymers.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” in this written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, an “active delivery” device is a device that delivers a therapeutic agent or payload with the assistance of an external force, current or the like. For example, electrophoresis, electroporation, sonophoresis, iontophoresis, etc.

The term “coupled,” as used herein, is defined as directly or indirectly connected in an electrical or nonelectrical manner. “Directly coupled” structures or elements are in physical contact with one another. Objects described herein as being “adjacent to” each other may be in physical contact with each other, in close proximity to each other, or in the same general region or area as each other, as appropriate for the context in which the phrase is used.

As used herein, comparative terms such as “increased,” “decreased,” “better,” “worse,” “higher,” “lower,” “enhanced,” “maximized,” “minimized,” “improved,” and the like refer to a property of a device, component, or activity that is measurably different from other comparable devices, components, or activities, or from different iterations or embodiments of the same device, properties lacking the same features or characteristics. For example, a reservoir with properties that provide “improved” drug release would achieve a result that is measurably different than a reservoir with different properties.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 milligrams to about 80 milligrams” should also be understood to provide support for the range of “50 milligrams to 80 milligrams.” Furthermore, it is to be understood that in this written description support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### EXAMPLE EMBODIMENTS

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

As discussed above, the eyes are subject to numerous adverse health conditions that can impair vision and eventually lead to blindness. Thus, effective administration of an active agent to the eye can be important in treating these ocular conditions in a timely manner. However, eye drops are often inefficient at delivering active agents to the eye, especially the posterior segment of the eye. As an alternative to eye drops, intraocular injections, intraocular implants, etc. are invasive administration procedures that can be more effective at delivering active agents to the posterior segment of the eye. However, invasive procedures also pose a number of significant risks, such as infection, retinal detachment, vitreous hemorrhage, etc. Thus, there is a need for non-invasive administration devices and methods that can provide an adequate dose of a therapeutic agent to the eye in a timely manner.

Accordingly, non-invasive ocular drug delivery devices are disclosed herein that can provide an adequate dose of an active agent to the eye in a timely manner. Generally, the non-invasive ocular drug delivery device can be a passive ocular drug delivery device. However, in some examples, the non-invasive ocular drug delivery device can be an active delivery device.

The non-invasive ocular drug delivery devices can include a housing adapted to couple to an eye of a subject. An active agent matrix can be coupled to the housing. The active agent matrix can include or be formed of an electrospun material having a combination of a density, a thickness, and an ocular surface area configured to hold and retain an active agent composition prior to application of the device to the eye, and then deliver an effective dose of an active agent within 30 minutes of application of the device to the eye.

In further detail, the housing of the non-invasive ocular drug delivery device is not particularly limited, other than it is adapted to couple to an eye of a subject. Thus, in some examples, the housing can couple directly to the eye, such as via negative pressure, surface tension, adhesives, the like, or combinations thereof. In yet other examples, the housing can be shaped to interface with the eye and can be merely held against the eye using positive pressure from eye lids, and/or straps, cords, scaffolding, adhesives, the like, or combinations thereof that are attached to a surface outside of the eye, but nonetheless hold the housing in place against the eye.

In some examples, the housing can be formed from a plurality of interconnecting pieces to prepare an integral housing. In yet other examples, the housing can be formed as a monolithic unit. Thus, in some cases, the housing can be formed from a mold or other suitable manufacturing process as a single monolithic unit without any need for further assembly or integration of additional components. In some specific examples, the monolithic unit can be formed of a molded elastomeric material, such as ethylene propylene diene monomer (EPDM), fluoroelastomers (e.g. FKMs, FFKMs, FEPMs, etc.), acrylonitrile-butadiene rubbers, silicones, the like, or combinations thereof. Whether the housing is formed of a molded material or not, the housing can include a variety of suitable materials, such as one or more of the elastomeric materials listed above, polyamides, polyesters, polyethylenes, polypropylenes, polycarbonates, polyurethanes, polytetrafluoroethylenes, metals, the like, or combinations thereof. In some specific examples, the housing can include or be formed of an EPDM material. In yet other examples, the housing can include or be formed of a fluoroelastomer material. In still other examples, the housing can include or be formed of an acrylonitrile-butadiene rubber. In yet additional examples, the housing can include or be formed of a silicone material.

In still additional examples, the housing can include or be formed of a translucent or transparent material. For example, many of the materials listed above can be prepared in a way so that they are translucent or transparent. Other translucent or transparent materials can also be used. In some examples, portions of the housing can be translucent or transparent while others are not. In yet other examples, portions of the housing can be translucent while other parts of the housing can be transparent. In some specific examples, at least a portion of the housing that covers the cornea can be translucent or transparent.

The geometry of the housing is not particularly limited, so long as the housing adequately interfaces with a surface of the eye to facilitate administration of an active agent. However, in some examples, the housing (or at least the portion of the housing that interfaces with the eye) can have an elliptical geometry. While the overall shape of the eye approaches a spherical geometry, the part of the eye that is visible generally has an elliptical shape. Thus, the housing (or at least the portion of the housing that interfaces with the eye) can be prepared so as to have an elliptical, or approximately elliptical, shape. In some examples, an elliptical shape can facilitate application of the device to the eye and maximize the comfort of the subject, while maintaining adequate surface coverage or interface area of the device with the eye to provide an adequate dose of an active agent in a timely manner. Where the device has an elliptical geometry, the device can typically have an aspect ratio (width to height) of from about 1.05:1 to about 1.4:1. In yet other examples, the device can have an aspect ratio of from about 1.10:1 to about 1.3:1. In still other examples, the device can have an aspect ratio of from about 1.15:1 to about 1.25:1.

In some specific examples, the housing can include a corneal dome shaped to cover the cornea of the eye. The corneal dome can generally be shaped to maintain a gap between a portion of the cornea and an inner surface of the corneal dome. This gap can also facilitate the comfort of the user while using the device. As is known to one skilled in the art, the cornea can be a very sensitive portion of the eye. As such, in some cases, it can facilitate user comfort by minimizing contact of the device with the cornea. In some examples, the gap between the portion of the cornea and the inner surface of the corneal dome can be at least 50 μm or at least 100 μm. In yet other examples, the gap between the portion of the cornea and the inner surface of the corneal dome can be at least 200 μm or at least 500 μm. In still other examples, the gap between the portion of the cornea and the inner surface of the corneal dome can be at least 1000 μm. The portion of the cornea where the gap is maintained can generally be at least 50% of the corneal surface area. Thus, for example, in some cases, a gap of at least 100 μm between an inner surface of the corneal dome and the cornea of the eye can be maintained over at least 50% of the corneal surface. In some examples, the portion of the cornea where the gap is maintained can be at least 60%, 70%, 80%, or 90% of the corneal surface area. In yet other examples, the gap can be maintained across the entire corneal surface area.

In some additional examples, the housing can include a corneal seal that is positioned to circumscribe the cornea and form a fluidic seal against the eye to minimize fluid transport across the corneal seal to the cornea when in use. It is noted that where the device does not include a corneal dome, the cornea can be exposed to ambient conditions. However, the corneal seal can still minimize fluid transport (e.g. from the active agent matrix, for example) across the surface of the eye to the cornea. Where the housing includes a corneal dome, the corneal seal can be disposed about a periphery of the corneal dome to minimize fluid transport to the cornea when in use. It is noted that when the diameter of the corneal seal becomes too large, it can be challenging to comfortably maintain the housing within the framework of the eyelids. Thus, the corneal seal can be shaped to maintain a seal about the cornea without excessively increasing the overall size of the housing. In some examples, the corneal seal can be shaped to maintain a distance from a perimeter of the cornea (i.e. the corneal seal is positioned exterior to the cornea so as to not contact the cornea) of from about 50 μm to about 5000 μm. In yet other examples, the corneal seal can be shaped to maintain a distance from a perimeter of the cornea of from about 500 μm to about 3000 μm. In still other examples, the corneal seal can be shaped to maintain a distance from a perimeter of the cornea of from about 1000 μm to about 2000 μm. In some specific examples, the corneal seal can be shaped to maintain a distance from a perimeter of the cornea of from about 50 μm to about 1000 μm, about 100 μm to about 1500 μm, or about 300 μm to about 1200 μm.

In some further examples, the housing can include a scleral flange extending radially outward from the corneal seal. In some examples, where the housing (or at least the portion of the housing that interfaces with the eye) has an elliptical geometry, the scleral flange can have a shape that provides the elliptical geometry. In some examples, the scleral flange can be the portion of the housing to which the active agent matrix is attached. Where this is the case, the scleral flange can be shaped to maintain contact between the active agent matrix and the sclera of the eye when in use. The scleral flange can generally be shaped and positioned on the housing so as to cover a portion of the sclera of the eye without covering the cornea. Additionally, in some examples, the scleral flange, or other similar segment of the housing, can include a scleral lip or scleral seal about a perimeter of the portion of the device that interfaces with the eye. In some examples, the scleral lip or scleral seal can be shaped to facilitate retention of the active agent matrix to the housing, such as via friction fitting, nesting, clamping, or the like. In some examples, the scleral lip or scleral seal can additionally form a fluidic seal against the eye to minimize fluid transport across the scleral seal. In some examples, this can help concentrate delivery of the active agent to a specific region of the sclera and improve delivery of the active agent to the posterior segment of the eye.

In some examples, a pressure regulator can be operatively connected to the housing and adapted to induce negative pressure between the housing and the eye to couple the housing to the eye when in use. In some examples, the pressure regulator can form part of the housing, such as an integrated component of the housing or as part of a monolithic housing. In some examples, the pressure regulator can be a bulb, a pump, the like, or other suitable pressure regulator that can be operatively connected to the housing. The pressure regulator can generally be adapted to induce a negative pressure between the housing and the eye to couple the housing to the eye when in use. The negative pressure induced between the housing and the eye can be any pressure suitable to maintain the housing on the eye without significantly damaging the eye. In some examples, the pressure regulator can be adapted to induce a negative pressure of from about 0.98 atmospheres (atm) to about 0.1 atm between the housing and the eye. In yet other examples, the pressure regulator can be adapted to induce a negative pressure of from about 0.90 atm to about 0.3 atm. In still other examples, the pressure regulator can be adapted to induce a negative pressure of from about 0.8 atm to about 0.5 atm. In some examples, the pressure regulator can be adapted to reduce a pressure between the housing and the eye by an amount from about 0.1 atm to about 3 atm relative to atmospheric pressure. In yet other examples, the pressure regulator can be adapted to reduce a pressure between the housing and the eye by an amount from about 0.5 atm to about 1 atm relative to atmospheric pressure.

The active agent matrix can be coupled to the housing using any suitable coupling feature, such as an adhesive, stitching, friction-fitting, clips, clamps, magnets, snaps, hook and loop fasteners, the like, or combinations thereof. In some specific examples, the active agent matrix can be coupled to the housing via an adhesive. A variety of suitable adhesives can be used. Non-limiting examples can include a silicone adhesive, an acrylic adhesive, a polyurethane adhesive, the like, or combinations thereof. Further, the active agent matrix can generally be positioned to interface with the sclera of the eye, but not the cornea of the eye. In some examples, the active agent matrix can be formed of a plurality of segments that are positioned adjacent to one another to form an integral active agent matrix. In some specific examples, the active agent matrix can be formed from 2, 3, 4, or more individual segments positioned adjacent to one another. In some examples, the individual segments can be spaced apart from one another. In yet other examples, the individual segments can be positioned so that there is substantially no space between adjacent segments.

As described above, in one embodiment the active agent matrix can be formed of an electrospun material. Electrospinning is a robust process capable of producing polymer fibers from a variety of polymer/solvent systems with diameters typically from about 100 nm to about 500 nm. Electrospinning can also be used to produce highly porous membranes with good structural integrity. Thus, electrospinning can be used to prepare a variety of materials that can be used as active agent reservoirs or matrices that have good structural integrity and porosity. It is noted that the porosity or surface area of an electrospun material can allow a solvent or an active agent composition to quickly absorb thereto. Similarly, an electrospun material can also allow an active agent composition to transfer quickly from the material once placed in contact with an ocular surface, without otherwise dripping or leaking prior to application. Further, in some examples, the electrospun material can be highly solvent-swellable, or highly solvent absorbable. For example, in some cases, the electrospun material or active agent matrix can absorb a solvent or an active agent composition so as to more than double the dry weight or pre-loaded weight of the electrospun material or active agent matrix. In other examples the electrospun material or active agent matrix can absorb a solvent or an active agent composition so as to increase the weight of the electrospun material or active agent matrix to from about 2 times to about 40 times the dry weight or pre-loaded weight of the electrospun material or active agent matrix. In yet other examples, the electrospun material or active agent matrix can absorb a solvent or an active agent composition so as to increase the weight of the electrospun material or active agent matrix to from about 3 times to about 30 times, from about 4 times to about 25 times, from about 5 times to about 20, or from about 6 times to about 10 or 15 times the dry weight or pre-loaded weight of the electrospun material or active agent matrix. In still other examples, the electro spun material or active agent matrix can absorb a solvent or an active agent composition so as to increase the weight of the electrospun material or active agent matrix to from about 2 times to about 9 times, or from about 3 times to about 7 times the dry weight or pre-loaded weight of the electrospun material or active agent matrix. In some specific examples, the electrospun material or active agent matrix can swell to accommodate absorbed solvent. In some additional specific examples, the solvent can be or include water.

In further detail, in some cases, the electrospun material can be a hydrophobic material. In yet other examples, the electrospun material can be a hydrophilic material. In some specific examples, the electrospun material can be a hydrogel material. However, the degree of hydrophobicity/hydrophilicity of the material can dependent on the active agent to be delivered and the carrier used to deliver the active agent. The electrospun material can be formed of a variety of materials. Non-limiting examples can include polyamides, polyurethanes, polycarbonates, polyvinyl alcohols, polylactic acids, polyglycolic acids, polyethylene-co-vinyl acetate, polyethylene oxide, polystyrene, collagen, polyvinylpyrrolidone, polyethylene, polypropylene, the like, or combinations thereof. In some examples, the active agent matrix can include or be formed of a non-biodegradable/non-bioabsorbable material. In some examples, the active agent matrix can include or be formed of a biodegradable/bioabsorbable material. In some specific examples, the electrospun material can be or include polyamide. In other examples, the electrospun material can be or include polyurethane. It is noted that where the material is or includes polyurethane, the polyurethane can be formulated as either a hydrophilic or hydrophobic variety of polyurethane depending on the ratios and types of isocyanate groups, polyols, chain extenders, etc. that are incorporated into the polyurethane polymer. Thus, in some examples the polyurethane can be a hydrophilic polyurethane, whereas in other examples the polyurethane can be a hydrophobic polyurethane. In some examples, the polyurethane can be a thermoplastic polyurethane. In some additional examples, the polyurethane can be a polyether-based polyurethane. In other examples, the polyurethane can be a polyester-based polyurethane. In other examples, the electrospun material can be or include polyvinyl alcohol. In other examples, the electrospun material can be or include polyvinylpyrollidone. In other examples, the electrospun material can be or include polylactic acid. In other examples, the electrospun material can be or include polyglycolic acid. In other examples, the electrospun material can be or include polycarbonate. In other examples, the electrospun material can be or include polyethylene. In other examples, the electrospun material can be or include polypropylene. In other examples, the electrospun material can be or include polyethylene oxide. In other examples, the electrospun material can be or include polystyrene. In other examples, the electrospun material can be or include collagen.

In addition to the specific composition of the electrospun material, the active agent matrix can have a combination of a density, a thickness, and an ocular surface area that is configured to hold and retain an active agent composition prior to application of the device to the eye, and deliver an effective dose of an active agent within 30 minutes of application of the device to the eye. By “hold and retain,” it is meant that the active agent matrix is configured to hold and retain a predetermined volume or threshold volume of a desired active agent composition without leaking, dripping, or otherwise prematurely releasing the active agent from the active agent matrix prior to application of the device to an eye of a subject. Thus, the active agent matrix can be configured to facilitate loading of the active agent composition to the active agent matrix, retaining the active agent composition without leaking, dripping, or otherwise prematurely releasing the active agent, and delivering the active agent within a predetermined delivery period of application of the device to the eye.

In further detail, the active agent matrix can have a variety of suitable densities. In some specific examples, the active agent matrix can have a density of from about 0.15 grams/cubic centimeter (cc) to about 0.4 grams/cc prior to loading with the active agent composition. In yet other examples, the active agent matrix can have a density of from about 0.18 g/cc to about 0.35 g/cc prior to loading the active agent composition. In still other examples, the active agent matrix can have a density of from about 0.2 g/cc to about 0.31 g/cc prior to loading the active agent composition.

The active agent matrix can also have a variety of thicknesses. In some specific examples, the active agent matrix can have a thickness of from about 250 μm to about 600 μm prior to loading with the active agent composition. In yet other examples, the active agent matrix can have a thickness of from about 300 μm to about 500 μm prior to loading with the active agent composition. In still other examples, the active agent matrix can have a thickness of from about 350 μm to about 450 μm prior to loading with the active agent composition. The post-loading thickness of the active agent matrix can typically be greater than the pre-loading thickness of the active agent matrix. For example, in some cases, the post-loading thickness can be from about 2 times to about 6 times the pre-loading thickness. In yet other examples, the post-loading thickness can be from about 3 times to about 5 times the pre-loading thickness.

The active agent matrix can have a variety of ocular surface areas or ocular interface areas (i.e. the area of the active agent matrix that interfaces with or otherwise faces toward the eye when the device/matrix is in use). As the active agent matrix is generally positioned so as to interface with the sclera while avoiding or minimizing contact with the cornea, the ocular surface area can be a scleral surface area or scleral interface area. In some examples, the ocular surface area of the active agent matrix can be from about 50 mm² to about 300 mm². In some additional examples, the ocular surface area of the active agent matrix can be from about 75 mm² to about 250 mm². In yet other examples, the ocular surface area of the active agent matrix can be from about 100 mm² to about 200 mm².

The combination of the density, thickness, and ocular surface area of the active agent matrix can generally provide a loading capacity to hold and retain at least 50 μL, at least 100 μL, or at least 150 μL of active agent composition. In some examples, the active agent matrix can have a loading capacity to hold and retain from about 50 μL to about 5000 μL of the active agent composition prior to application. In other examples, the active agent matrix can have a loading capacity to hold and retain from about 100 μL to about 1000 μL of the active agent composition prior to application. In yet other examples, the active agent matrix can have a loading capacity to hold and retain from about 150 μL to about 500 μL of the active agent composition prior to application. In some specific examples, the active agent matrix can have a loading capacity to hold and retain from about 120 μL to about 300 μL of active agent composition prior to application.

It is noted, that the active agent matrix can hold and retain at least 99%, at least 98%, at least 95%, or at least 90% of a target volume of the active agent composition loaded thereto for a period of at least 30 seconds. In some examples, the active agent matrix can hold and retain at least 99%, at least 98%, at least 95%, or at least 90% of a target volume of the active agent composition loaded thereto for a period of at least 60 seconds. In other examples, the active agent matrix can hold and retain at least 99%, at least 98%, at least 95%, or at least 90% of a target volume of the active agent composition loaded thereto for a period of at least 5 minutes. In yet other examples, the active agent matrix can hold and retain at least 99%, at least 98%, at least 95%, or at least 90% of a target volume of the active agent composition loaded thereto for a period of at least 10 minutes. In still other examples, the active agent matrix can hold and retain at least 99%, at least 98%, at least 95%, or at least 90% of a target volume of the active agent composition loaded thereto for a period of at least 30 minutes, at least 60 minutes, or at least 120 minutes. This assumes that the active agent matrix is not interfaced with a surface, material, or medium similar to the active agent matrix or otherwise configured to similarly or preferentially hold and retain the active agent composition as compared to the active agent matrix.

It is further noted that, in some examples, the active agent composition can be pre-loaded into the active agent matrix and sealed in a container to provide added retentiveness or shelf-life for the active agent composition in the matrix. In some examples, the pre-loaded active agent matrix can be covered by a release liner, interfaced with an impermeable or minimally permeable surface, or the like to help maximize the retention of the active agent composition within the active agent matrix. In some examples, where this is the case, the active agent matrix can hold and retain a target volume, or at least 95%, 90%, or 80% of the target volume, of the active agent composition for a period of at least 1 month at ambient temperature without leaking, dripping, or otherwise prematurely releasing the active agent composition. In additional examples, the active agent matrix can hold and retain a target volume, or at least 95%, 90%, or 80% of the target volume, of the active agent composition for a period of at least 3 months at ambient temperature without leaking, dripping, or otherwise prematurely releasing the active agent composition. In other examples, the active agent matrix can hold and retain a target volume, or at least 95%, 90%, or 80% of the target volume, of the active agent composition for a period of at least 6 months at ambient temperature without leaking, dripping, or otherwise prematurely releasing the active agent composition. One way to measure the pre-application retentiveness of the active agent matrix for the active agent composition is to load the active agent matrix with the target volume of the active agent composition and record the weight. After a predetermined period of time (e.g. 1 month, 3 months, 6 months, etc.), the active agent matrix can be weighed again to determine any changes in mass over time.

In some examples, the target volume can be from about 50 μL to about 5000 μL of the active agent composition. In other examples, the target volume can be from about 100 μL to about 1000 μL of the active agent composition. In yet other examples, the target volume can be from about 150 μL to about 500 μL of the active agent composition. In still other examples, the target volume can be from about 120 μL to about 300 μL of active agent composition.

Additionally, the composition of the active agent matrix in combination with the density, thickness, and ocular surface area of the active agent matrix can typically facilitate rapid loading of the active agent composition into the active agent matrix. Thus, the composition of the active agent matrix and the formulation of the active agent composition can be sufficiently compatible to facilitate rapid loading (e.g. hydrophilic matrix and hydrophilic formulation, hydrophobic matrix and hydrophobic formulation, etc.) For example, in some cases, the active agent matrix can be configured to passively absorb at least 100 μL, at least 120 μL, or at least 150 μL of the active agent composition within 10 minutes. In yet other examples, the active agent matrix can be configured to passively absorb at least 100 μL, at least 120 μL, or at least 150 μL of the active agent composition within 5 minutes. In still additional examples, the active agent matrix can be configured to passively absorb at least 100 μL, at least 120 μL, or at least 150 μL of the active agent composition within 2 minutes.

Further, the active agent matrix can be sufficiently stable to maintain rapid loading of the active agent composition after storage of the active agent matrix in a dry state at ambient conditions for a prolonged period, and optionally after sterilization of the active agent matrix. Specifically, in some examples, the active agent matrix can be configured to passively absorb at least 100 μL, at least 120 μL, or at least 140 μL of the active agent composition within 2 minutes after a storage period of at least 3 months in a dry state and at ambient conditions. In some additional examples, the active agent matrix can be configured to passively absorb at least 100 μL, at least 120 μL, or at least 140 μL of the active agent composition within 2 minutes after a storage period of at least 6 months in a dry state and at ambient conditions. In other examples, the active agent matrix can be configured to passively absorb at least 100 μL, at least 120 μL, or at least 140 μL of the active agent composition within 2 minutes after a storage period of at least 12 months in a dry state and at ambient conditions. In yet other examples, the active agent matrix can be configured to passively absorb at least 100 μL, at least 120 μL, or at least 140 μL of the active agent composition within 2 minutes after a storage period of at least 24 months in a dry state and at ambient conditions.

Moreover, the composition of the active agent matrix in combination with the density, thickness, and ocular surface area of the active agent matrix can typically facilitate rapid delivery of an effective dose of the active agent to the eye. In some examples, the active agent matrix can be configured to deliver the effective dose within 30 minutes. In other examples, the active agent matrix can be configured to deliver the effective dose within 20 minutes. In yet other examples, the active agent matrix can be configured to deliver the effective dose within 10 minutes. In still other examples, the active agent matrix can be configured to deliver the effective dose within 5 minutes.

In some examples, the effective dose is from about 5 wt % to about 50 wt % of the active agent loaded into the active agent matrix. Thus, for example, where 10 mg of active agent is loaded into the active agent matrix, in some cases, the effective dose can be from 0.5 mg to 5 mg of that active agent. In other examples, the effective dose is from about 1 wt % to about 20 wt % of the active agent loaded into the active agent matrix. In yet other examples, the effective dose is from about 8 wt % to about 40 wt % of the active agent loaded into the active agent matrix. In still other examples, the effective dose is from about 10 wt % to about 30 wt % of the active agent loaded into the active agent matrix.

In some examples, the effective dose can be an amount from about 0.01 mg to about 100 mg of the active agent. In other examples, the effective dose can be an amount from about 0.05 mg to about 0.1 mg. In yet other examples, the effective dose can be an amount from about 0.1 mg to about 1 mg. In additional examples, the effective dose can be an amount from about 1 mg to about 10 mg, from about 5 mg to about 15 mg, or from about 10 mg to about 20 mg. In still other examples, the effective dose can be an amount from about 20 mg to about 100 mg. In yet other examples, the effective dose can be from about 0.1 mg to about 0.5 mg. In still other examples, the effective dose can be an amount from about 0.2 mg to about 0.4 mg.

In some examples, the effective dose can be from about 5 vol % to about 50 vol % of the active agent composition or target volume loaded into the active agent matrix. Thus, for example, where a target volume of the active agent composition of 100 μL is loaded into the active agent matrix, in some cases, the effective dose can be from 5 μL to 50 μL of that active agent composition. In other examples, the effective dose is from about 1 vol % to about 20 vol % of the active agent composition or target volume loaded into the active agent matrix. In yet other examples, the effective dose is from about 8 vol % to about 40 vol % of the active agent composition or target volume loaded into the active agent matrix. In still other examples, the effective dose is from about 10 vol % to about 30 vol % of the active agent composition or target volume loaded into the active agent matrix.

Further, the active agent matrix can be sufficiently stable to maintain rapid delivery of an active agent composition that is subsequently loaded to the active agent matrix after storage of the active agent matrix in a dry state at ambient conditions for a prolonged period, and optionally after sterilization of the active agent matrix. In some specific examples, the active agent matrix can be configured to passively deliver an effective dose of at least 10 wt %, at least 20 wt %, or at least 40 wt % of the loaded active agent composition within 10 minutes of application after a storage period of at least 3 months in a dry state and at ambient conditions. In some additional examples, the active agent matrix can be configured to passively deliver am effective dose of at least 10 wt %, at least 20 wt %, or at least 40 wt % of the loaded active agent composition within 10 minutes of application after a storage period of at least 6 months in a dry state and at ambient conditions. In other examples, the active agent matrix can be configured to passively deliver an effective dose of at least 10 wt %, at least 20 wt %, or at least 40 wt % of the loaded active agent composition within 10 minutes of application after a storage period of at least 12 months in a dry state and at ambient conditions. In yet other examples, the active agent matrix can be configured to passively deliver an effective dose of at least 10 wt %, at least 20 wt %, or at least 40 wt % of the loaded active agent composition within 10 minutes of application after a storage period of at least 24 months in a dry state and at ambient conditions.

The effective dose can depend on the active agent employed and the type/severity of the condition being treated. The non-invasive ocular delivery device can be used to deliver a large range of active agents. Generally, any active agent that is suitable for topical administration to the eye can be used in the non-invasive ocular drug delivery device. Non-limiting examples can include a steroid, an antimicrobial agent, an immunosuppressive agent, a non-steroidal anti-inflammatory agent, an anti-angiogenic agent, a vasoconstrictive agent, an antihistamine, a glaucoma agent (e.g. a prostaglandin, a beta-blocker, an alpha-adrenergic agonist, a carbonic anhydrase inhibitor), an anesthetic, an analgesic, the like, or a combination thereof. In some examples, where the active agent is a macromolecular active agent, the non-invasive ocular delivery device can be adapted as an active delivery device. Otherwise, the non-invasive ocular delivery device can generally be a passive delivery device. As such, in some examples, the non-invasive ocular delivery device does not include an electrode or other electrical components used in an active delivery device. In some specific examples, the non-invasive ocular delivery device does not include an electrode or other electrical components adapted specifically for iontophoretic administration of the active agent. In other examples, the non-invasive ocular drug delivery device can include an electrode and/or other electrical components to adapt the device for active administration of the active agent, such as iontophoretic delivery, electroporation, sonoporation, the like, or combinations thereof.

In some examples, the active agent can be or include a steroid. Where this is the case, the active agent can include fluocinolone, difluprednate, fluorometholone, loteprednol, dexamethasone, prednisolone, medrysone, triamcinolone, rimexolone, the like, a salt thereof, an ester thereof, a hydrate thereof, derivatives thereof, or a combination thereof. In some specific examples, the active agent can be or include dexamethasone sodium phosphate (DSP). In other examples, the active agent can be or include triamcinolone acetonide sodium phosphate.

In some examples, the active agent can be or include an antimicrobial agent. Where this is the case, in some examples, the active agent can include an antibacterial agent, such as besifloxacin, ciprofloxacin, levofloxacin, ofloxacin, moxifloxacin, gatifloxacin, tobramycin, gentamicin, polymyxin B, trimethoprim, bacitracin, neomycin, gramicidin, azithromycin, erythromycin, the like, salts thereof, esters thereof, a hydrate thereof, derivatives thereof, or combinations thereof. In some examples, the active agent can include an antiviral agent, such as fluorometholone, ganciclovir, trifluridine, the like, salts thereof, esters thereof, a hydrate thereof, derivatives thereof, or combinations thereof. In some examples, the active agent can include an antifungal agent, such as clotrimazole, econazole, ketoconazole, miconazole, bifonazole, isoconazole, neticonazole, sertaconazole, fluconazole, fosfluconazole, itraconazole, posaconazole, voriconazole, thiabendazole, nystatin, amphotericin B, natamycin, terbinafine, butenafine, amorolfine, caspofungin, micafungin, anidulafungin, flucytosine, gresiofulvin, the like, salts thereof, esters thereof, a hydrate thereof, derivatives thereof, or combinations thereof.

In some examples, the active agent can be or include an immunosuppressive agent. Where this is the case, the active agent can include cyclophosphamide, chlorambucil, azathioprine, methotrexate, mycophenolic acid, cyclosporine, tacrolimus, infliximab, adalimumab, rapamycin, the like, salts thereof, esters thereof, a hydrate thereof, derivatives thereof, or combinations thereof.

In some examples, the active agent can be or include a non-steroidal anti-inflammatory agent. Where this is the case, the active agent can include ketorolac tromethamine, flurbiprofen, diclofenac, bromfenac, nepafenac, the like, salts thereof, esters thereof, a hydrate thereof, derivatives thereof, or combinations thereof.

In some examples, the active agent can be or include an anti-angiogenic agent. Wherein this is the case, the active agent can include ranibizumab, bevacizumab, pegaptanib, aflibercept, the like, salts thereof, esters thereof, a hydrate thereof, derivatives thereof, or combinations thereof.

In some examples, the active agent can include a vasoconstrictive agent. Wherein this is the case, the active agent can include naphazoline, tetrahydrozoline, phenylethylamine, epinephrine, norepinephrine, dopamine, dobutamine, colterol, ethylnorepinephrine, isoproterenol, isotharine, metaproterenol, terbutaline, metearaminol, phenylephrine, tyramine, hydroxyamphetamine, ritodrine, prenalterol, methoxyamine, albuterol, amphetamine, methamphetamine, benzphetamine, ephedrine, phenylpropanolamine, methentermine, phentermine, fenfluramine, propylhexedrine, diethylpropion, phenmetrazine, phendimetrazine, the like, salts thereof, esters thereof, a hydrate thereof, derivatives thereof, or combinations thereof.

In some examples, the active agent can be or include an antihistamine. Where this is the case, the antihistamine can include emedastine difumarate, epinastine, azelastine, ketotifen, olopatadine, bepotastine, alcaftadine, cetirizine, chlorpheniramine maleate, the like, salts thereof, esters thereof, a hydrate thereof, derivatives thereof, or combinations thereof.

In some examples, the active agent can be or include a glaucoma agent. Where this is the case, the glaucoma agent can include timolol, brimonidine, brinzolamide, travoprost, tafluprost, dorzolamide, apraclonidine, latanoprost, bimatoprost, levobunolol, betaxolol, carbachol, epinephrine, physostigmine, carbachol, pilocarpine, acetylcholine, carbachol, carteolol, metipranolol, echothiophate iodide, dipivefrin, unoprostone, the like, salts thereof, esters thereof, a hydrate thereof, derivatives thereof, or combinations thereof.

In some examples, the active agent can be or include an anesthetic. Where this is the case, the anesthetic can include lidocaine, proparacaine, tetracaine, bupivacaine, benoxinate, the like, salts thereof, esters thereof, a hydrate thereof, derivatives thereof, or combinations thereof.

In some examples, the active agent can be or include an analgesic. Where this is the case, the analgesic can include a steroid listed above, a non-steroidal anti-inflammatory agent listed above, an immunomodulator (e.g. cyclosporine, dapsone, tacrolimus, sirolimus, mitomycin, antilymphocyte serum, anti-T cell antibody, gamma globulin, cyclophosphamide, chlorambucil, methotrexate, 5-fluorouracil, azathioprine, or the like), an opioid (e.g. codeine, morphine, oripavine, pseudomorphine, thebaine, a morphinan, a benzomorphan, a pethidine, a prodine, a ketobemidone, an amidone, a methadol, a moramide, a thiabutene, a phenalkoxam, an ampromide, an anilidopiperidine, an oripavine, a phenazepane, a priintramide, a benzimidazole, an indole, a beta-amino ketone, a diphenylmethylpiperazine, derivatives thereof, etc.), the like, salts thereof, esters thereof, a hydrate thereof, derivatives thereof, or combinations thereof.

The active agent can be present in the active agent composition in various amounts, depending on the desired dosage, the specific active agent being employed, the condition to be treated, etc. In some examples, the active agent can be present in the active agent composition in an amount from about 0.005 w/v % to about 25 w/v %. In other examples, the active agent can be present in the active agent composition in an amount from about 0.0001 w/v % to about 1 w/v %. In yet other examples, the active agent can be present in the active agent composition in an amount from about 0.01 w/v % to about 10 w/v %. In some specific examples, the active agent can be present in the active agent composition in an amount from about 1 w/v % to about 10 w/v %, from about 5 w/v % to about 15 w/v %, or from about 10 w/v % to about 20 w/v %.

The active agent composition can also vary depending on the particular active agent being administered. In some examples, the active agent composition can be a hydrophilic composition. In other examples, the active agent composition can be a lipophilic composition. In some examples, the active agent composition can be an emulsion, such as an oil-in-water emulsion or a water-in-oil emulsion. In some examples, the active agent composition can include micelles, liposomes, molecular carriers that are charged or soluble in water but that can be loaded with water-insoluble active agents (e.g. cyclodexrins, etc.), the like, or combinations thereof. In some specific examples, the active agent composition can include water. In some examples, the active agent composition can be substantially free of solvents other than water. In some examples, the active agent composition can include a lubricant such as polyethylene glycol (PEG) (e.g. PEG-200, PEG-300, PEG-400), propylene glycol, glycerin, mineral oil, the like, or combinations thereof. In some additional examples, the active agent composition can include a preservative, such as benzalkonium chloride, cetrimonium, chlorobutanol, polyquarternium-1, polyhexamethylene biguanide, sodium perborate, stabilized oxychloro complex, the like, or combinations thereof. In some examples, the active agent composition can include a chelating agent, such as edetate disodium dihydrate, edetic acid, ethylene diamine, porphine, the like, or combinations thereof. In some examples, the active agent composition can include phosphate-buffered saline (PBS), Dulbecco's PBS, Alsever's solution, Tris-buffered saline (TBS), water, balanced salt solutions (BSS), such as Hank's BSS, Earle's BSS, Grey's BSS, Puck's BSS, Simm's BSS, Tyrode's BSS, BSS Plus, Ringer's lactate solution, normal saline (i.e. 0.9% saline), ½ normal saline, the like, or a combination thereof.

In further detail, the active agent composition can generally have a pH of from about 5 to about 8. In some specific examples, the active agent composition can have a pH of from about 6 to about 8, or from about 6.5 to about 7.5. Suitable pH adjusters can be used to adjust the pH, which can include a number of acids, bases, and combinations thereof, such as hydrochloric acid, phosphoric acid, citric acid, sodium hydroxide, potassium hydroxide, calcium hydroxide, and the like

Additionally, the active agent composition can typically have a tonicity of from about 250 milliosmoles (mOsm)/kilogram (kg) to about 750 mOsm/kg. In some specific examples, the active agent composition can have a tonicity of from about 250 mOsm/kg to about 450 mOsm/kg, or from about 450 mOsm/kg to about 750 mOsm/kg. In some additional examples, the active agent composition can have a tonicity of from about 250 mOsm/kg to about 350 mOsm/kg, or from about 277 mOsm/kg to about 310 mOsm/kg. In some examples, the active agent composition can include a tonicity agent, such as sodium chloride, potassium chloride, calcium chloride, magnesium chloride, mannitol, sorbitol, dextrose, glycerin, propylene glycol, ethanol, trehalose, the like, or combinations thereof.

The active agent composition can typically be particulate-matter-free or substantially particulate-matter-free. As used herein, the term “particulate-matter-free” or its grammatical equivalents such as “particle free” refer to the state in which the active agent composition meets the USP requirements for particulate matter in ophthalmic compositions. See for example, USP, Chapter 789. One of skill in the art understands and knows how to assess whether a given composition meets USP particulate matter requirements.

In some examples, the non-invasive ocular drug delivery device and/or the active agent composition can be sterile. A number of sterilization procedures can be used to sterilize the device and/or the active agent composition. Non-limiting examples of sterilization procedures can include EtO sterilization, gamma sterilization, E-beam sterilization, x-ray sterilization, vaporized hydrogen peroxide (VHP) sterilization, steam sterilization, dry-heat sterilization, filtration, the like, or combinations thereof. In some examples, the active agent composition can be pre-loaded into the non-invasive ocular delivery device. Where this is the case, it is possible to terminally sterilize the device and the composition. In yet other examples, the active agent composition can be pre-loaded into the device, but the composition and the device can be individually or aseptically sterilized. In yet other examples, the active agent composition can be loaded into the device at the time of use. In such examples, the composition and device are typically sterilized individually or aseptically.

In some examples, to facilitate loading of the non-invasive ocular drug delivery device, the device can include a loading base or dock. The loading base can be shaped to removably mate with or engage the housing of the device to facilitate loading of the active agent composition into the active agent matrix. In some examples, the loading base can include a port to introduce the active agent composition into or onto the loading base. In some further examples, the loading base can include a reservoir, basin, or channel fluidly connected to the port to collect and hold the active agent composition prior to and/or during loading of the active agent composition into the non-invasive ocular drug delivery device. The reservoir, basin, or channel can be positioned to interface with the active agent matrix to facilitate passive absorption of the active agent composition into the active agent matrix of the device.

The present disclosure also describes a method of manufacturing a non-invasive ocular drug delivery device. The method can include preparing a housing that is adapted to couple to an eye of a subject. The method can further include forming an active agent matrix from an electrospun material. The active agent matrix can have a combination of a density, a thickness, and an ocular surface area configured to hold and retain an active agent composition prior to application of the device to the eye. Further, the combination of the density, the thickness, and the ocular surface area can be configured to deliver an effective dose of the active agent within 30 minutes of application of the device to the eye. The active agent matrix can be coupled to the housing in any suitable way.

The present disclosure also describes a method of treating a subject with an ocular condition. The method can include coupling a non-invasive ocular drug delivery device, as described herein, to an eye of a subject. The method can further include passively administering an active agent from the device to an eye of the subject during a continuous dosing period to provide a threshold dose of the active agent to the eye within 30 minutes of coupling the device to the eye.

The method of treating a subject can be used to treat a variety of ocular conditions. Generally any ocular disease or condition that can be reasonably treated with the present device and method is considered within the scope of the present disclosure. Non-limiting examples can include uveitis, age-related macular degeneration (AMD), macular edema, a cataract, diabetic retinopathy, glaucoma, dry eye, post-operation inflammation, eye infection, allergic conjunctivitis, presbyopia, corneal wound healing, ocular pain, the like, or combinations thereof.

The device can be adapted to continuously deliver active agent to the eye during the entire duration of the continuous dosing period. As such, the active agent matrix can typically include an excess of the active agent composition and active agent, although this is not required. Thus, in some cases, the method can be adapted to terminate passive delivery of the threshold dose at a predetermined time point by removing the non-invasive ocular drug delivery device from the eye. In some examples, the threshold dose can include a sufficient amount of the active agent to deliver the active agent to the posterior segment of the eye, such as the retina, choroid, vitreous humor, optic nerve, or a combination thereof. Therefore, the method can be adapted to deliver active agent to both the anterior and posterior segments of the eye via topical, passive administration of the active agent to the surface of the eye. In some examples, passive administration can deliver the active agent to the eye via the sclera. In some examples, passive administration can minimize delivery of the active agent to the eye via the cornea.

In some examples, the method can also include administering an anesthetic to the eye concurrently with or prior to coupling the non-invasive ocular drug delivery device to the eye of the subject. In some cases, this can increase the comfort of the subject during treatment. A variety of suitable anesthetics can be used. Non-limiting examples can include lidocaine, proparacaine, tetracaine, the like, or combinations thereof.

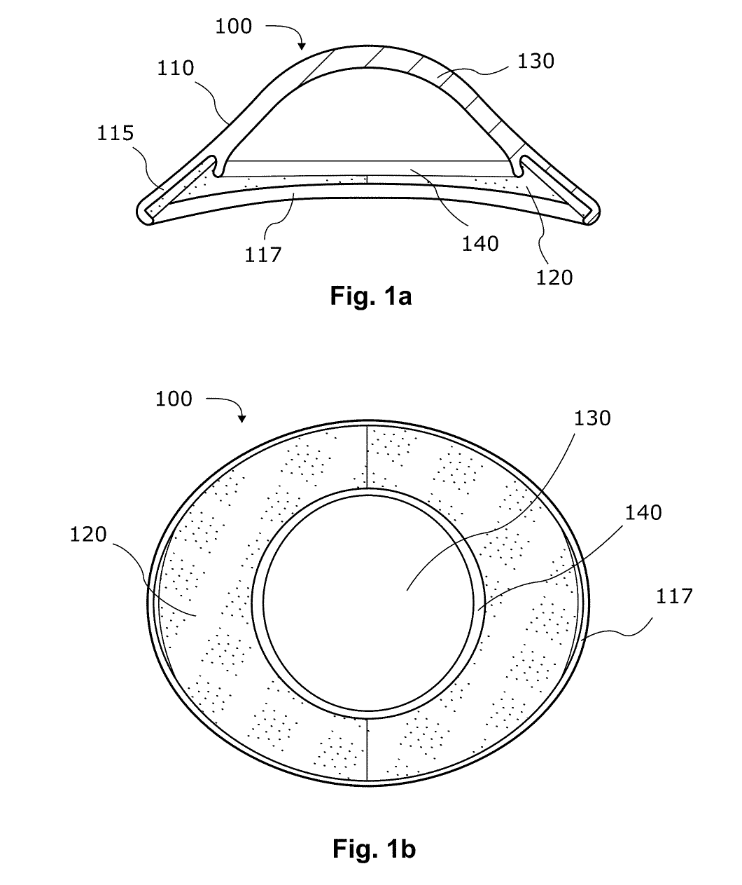

Turning now to the figures, FIGS. 1a and 1b illustrate one example of a non-invasive ocular drug delivery device 100 having a housing 110 and an active agent matrix 120 coupled thereto. In this particular example, the active agent matrix 120 includes two semicircle segments, but can include a single segment or other suitable number of segments. The housing 100 includes a corneal dome 130 shaped to cover a cornea of an eye. Additionally, the housing includes a corneal seal 140 positioned about a perimeter of the corneal dome 130 to form a fluidic seal against the eye when in use to minimize fluid transport into the corneal dome 130. The housing also includes a scleral flange 115 positioned to cover a portion of the sclera of an eye without covering the cornea. A scleral lip or scleral seal 117 is disposed about a perimeter of the scleral flange 115.

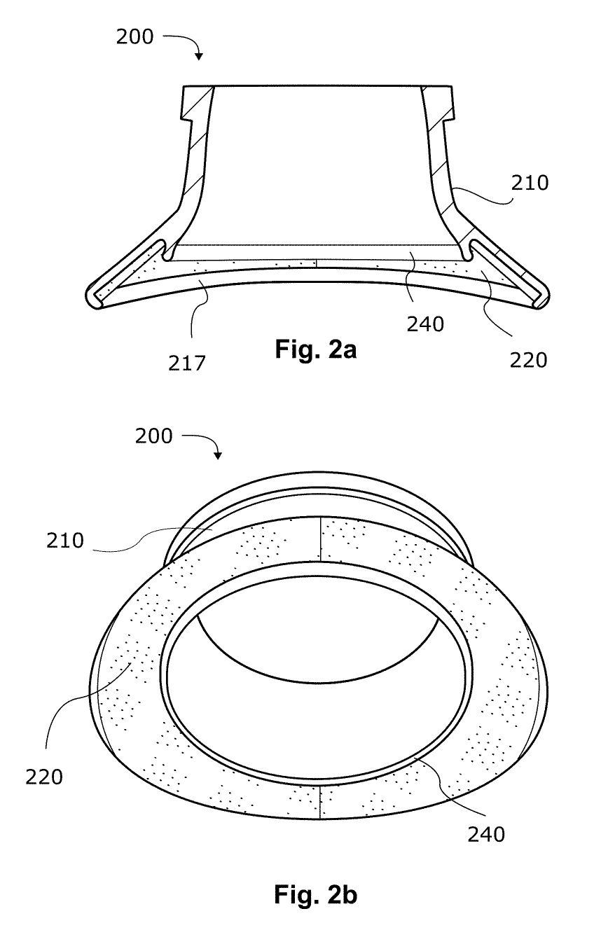

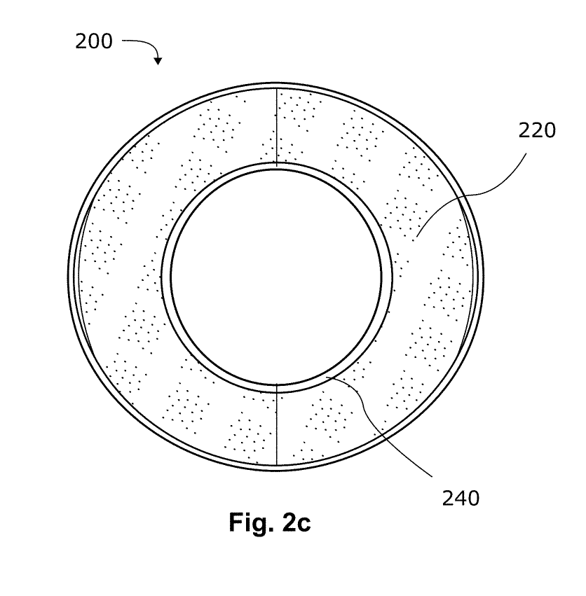

FIGS. 2a, 2b, and 2c illustrate an alternative example of a non-invasive ocular drug delivery device 200 having a housing 210 and an active agent matrix 220 coupled thereto. In this particular example, the housing 200 does not include a corneal dome. As such, the cornea of the eye can be exposed to ambient conditions during use of this particular example of the device 200. Nonetheless, the device 200 still includes a corneal seal 240 to minimize fluid transport across the surface of the eye to the cornea. This can minimize surface contact of the active agent with the sensitive cornea. The device 200 can also include a scleral lip or scleral seal 217 adapted to contain topical delivery of the active agent between the corneal seal 240 and the scleral seal 217.

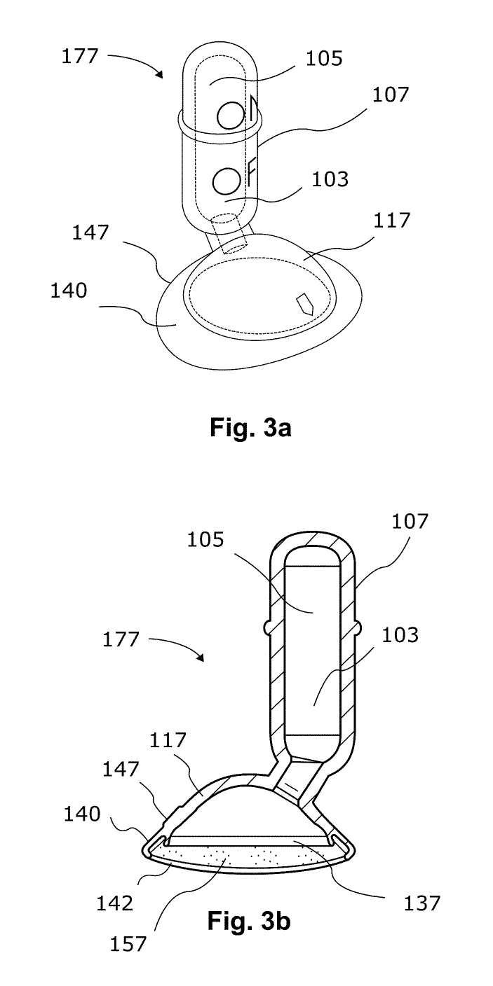

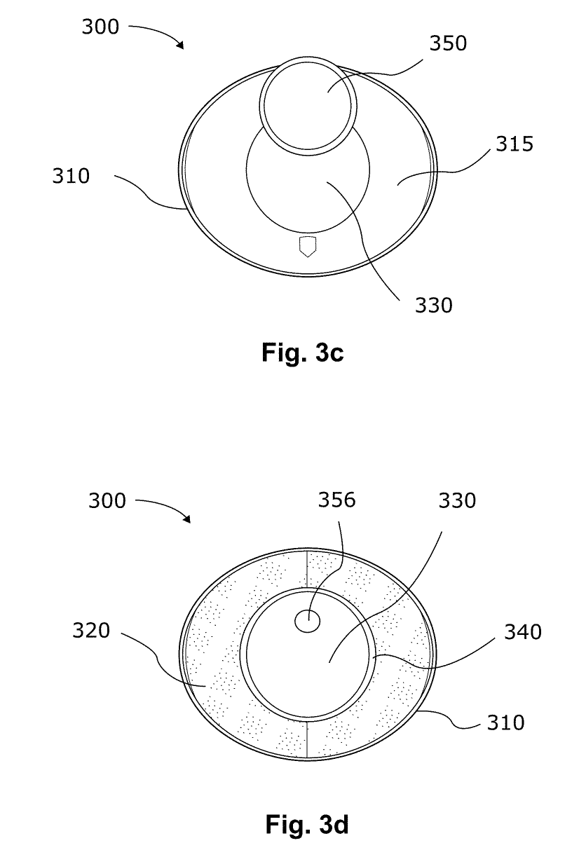

FIGS. 3a, 3b, 3c, and 3d illustrate yet another example of a non-invasive ocular delivery drug device 300. In this example, the device 300 includes a housing 310 with an active agent matrix 320 coupled thereto. Additionally, a pressure regulator 350 is coupled to a corneal dome 330 of the housing via pressure channel 356 to induce negative pressure between the housing and the eye. In this particular example, the negative pressure can be isolated to the corneal region of the device because the device includes a corneal dome 330 and a corneal seal 340 to maintain the pressure within the corneal region of the device. Additionally, in this particular example, the pressure regulator 350 can be marked, or include instructions, for applying device 300 to the eye and removing the device 300 from the eye. For example, segment 352 of the pressure regulator 350 can be marked for placement of device 300 on the eye, whereas segment 354 can be marked for removal of device 300 from the eye. In some examples, the segment 352 can form a lesser volume of the pressure regulator 350 than segment 354. As such, depressing segment 352 prior to application of the device 300 to the eye can generate sufficient negative pressure between the eye and the device 300 to couple the device 300 to the eye when segment 352 is released. Conversely, segment 354 can form a greater volume of the pressure regulator 350 than segment 352. As such, when it is desirable to remove the device 300 from the eye, depression of segment 354 can induce sufficient positive pressure between the device 300 and the eye to facilitate removal of the device 300 from the eye.

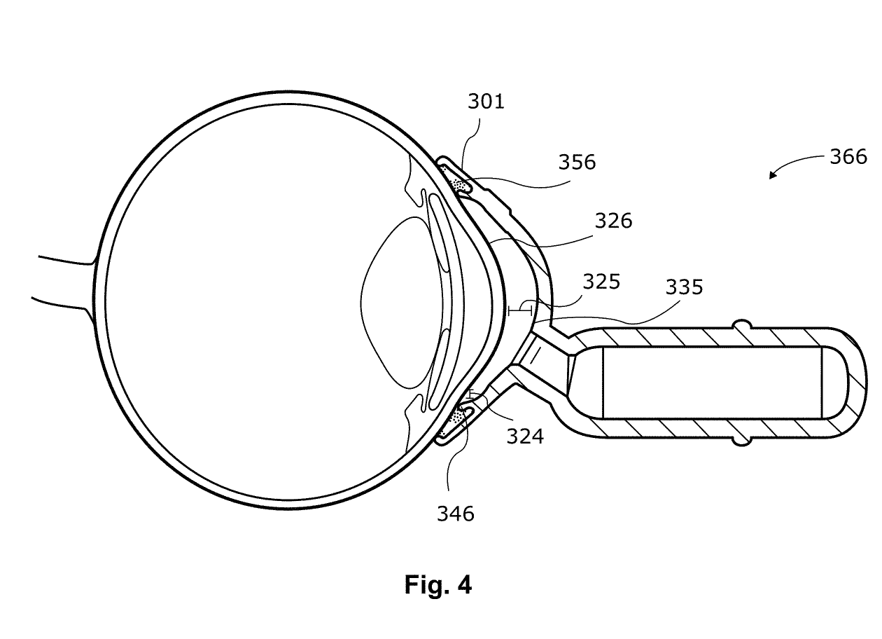

FIG. 4 illustrates an example of the device 300 coupled to an eye. As can be seen in this particular figure, a gap 362 can be maintained between an inner surface 332 of the housing and the cornea 360 so as to minimize contact of the housing 310 with the cornea 360. Additionally, a distance 364 can be maintained between the perimeter of the cornea 360 and the corneal seal 340 so as to maintain a fluidic seal about the cornea and minimize fluid transport across the surface of the eye to the cornea 360.

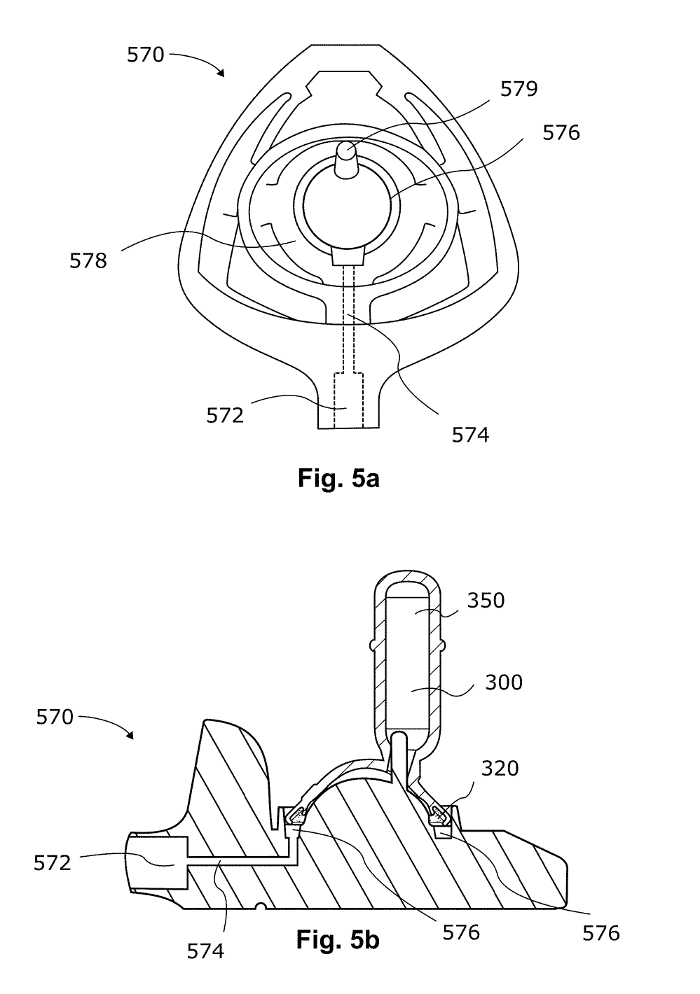

FIGS. 5a and 5b illustrate one example of a loading base 570 to facilitate loading of a non-invasive ocular drug delivery device 300. In this particular example, the loading base 570 can include an injection or infusion port 572 in fluid communication with a loading reservoir 576 via channel 574. The active agent composition can thus be loaded into the loading reservoir 576 by injecting the composition into the loading base 570 at the injection port 572. In some examples, the loading base 570 can include a guide post 579 and/or docking basin 578 to facilitate placement of the non-invasive ocular drug delivery device 300 on the loading base. Thus, in some examples, the docking basin 578 can mate with an ocular-interfacing portion of the non-invasive ocular drug delivery device 300 so as to facilitate loading of the active agent composition to the active agent matrix 320. Further, in some examples, the loading reservoir 576 can also flood upward into the docking basin 578 to expedite loading of the active agent matrix 320. In some examples, where the non-invasive ocular drug delivery device 300 includes a pressure regulator 350 or a port for attachment of a pressure regulator, the guide post 579 to mate with the pressure regulator 350 or associated port to facilitate intended positioning of the non-invasive ocular drug delivery device 300 on the loading base 570.

The following presents a few non-limiting illustrative examples of the present devices and methods:

In some examples, a non-invasive ocular drug delivery device can include a housing configured to couple to an eye of a subject and an active agent matrix coupled to the housing, said active agent matrix comprising an electrospun material having a combination of a density, a thickness, and an ocular surface area configured to hold and retain an active agent composition prior to application of the device to the eye, and deliver an effective dose of an active agent within 30 minutes of application of the device to the eye.

In some examples, the non-invasive ocular drug delivery device is a passive drug delivery device.

In some examples, the housing is a monolithic unit.

In some examples, the monolithic unit is formed from a molded elastomeric material.

In some examples, the housing is formed from a translucent material.

In some examples, the portion of the housing that interfaces with the eye, or otherwise faces the eye during use, has an elliptical geometry.

In some examples, the elliptical geometry has an aspect ratio (width to height) of from about 1.05:1 to about 1.4:1.

In some examples, the housing comprises a corneal dome shaped to cover a cornea of the eye.

In some examples, the corneal dome is further shaped to maintain a gap between a portion of the cornea and an inner surface of the corneal dome.

In some examples, the gap between the cornea and the inner surface of the corneal dome is at least 100 μm.

In some examples, the portion of the cornea is at least 50% of the corneal surface area.

In some examples, the housing comprises a corneal seal disposed about a periphery of the corneal dome to circumscribe the cornea and form a fluidic seal against the eye to minimize fluid transport into the corneal dome when in use.

In some examples, the corneal seal is shaped to maintain a distance from a perimeter of the cornea of from about 50 μm to about 5000 μm.

In some examples, the housing comprises a scleral flange extending radially outward from the corneal seal.

In some examples, the device can further include a pressure regulator operatively connected to the housing and adapted to induce negative pressure between the housing and the eye to couple the housing to the eye when in use.

In some examples, the pressure regulator forms part of the housing.

In some examples, the pressure regulator is adapted to reduce a pressure between the housing and the eye by an amount from about 0.1 atmospheres (atm) to about 3 atm relative to atmospheric pressure.

In some examples, the active agent matrix is positioned to interface with a sclera of the eye, but not a cornea of the eye.

In some examples, the active agent matrix is coupled to the housing via an adhesive.

In some examples, the adhesive is a member selected from the group consisting of: a silicone adhesive, an epoxy adhesive, an acrylic adhesive, a polyurethane adhesive, and combinations thereof.

In some examples, the electrospun material is a hydrophobic material.

In some examples, the electrospun material is a hydrophilic material.

In some examples, the electrospun material is a polyurethane material.

In some examples, the active agent matrix is solvent-swellable to a weight of from about 2 times to about 9 times the dry weight of the active agent matrix.

In some examples, the density of the active agent matrix is from about 0.15 grams/cubic centimeter (cc) to about 0.4 grams/cc prior to loading.

In some examples, the thickness of the active agent matrix is from about 250 μm to about 600 μm prior to loading.

In some examples, the ocular surface area of the active agent matrix is from about 50 mm² to about 300 mm².

In some examples, the active agent matrix has a loading capacity to hold and retain from about 50 μL to about 5000 μL of the active agent composition prior to application.

In some examples, the active agent matrix is configured to passively absorb at least 100 μL of the active agent composition within 10 minutes.

In some examples, the active agent matrix is configured to deliver the effective dose of the active agent within 20 minutes of application of the device to the eye.

In some examples, the effective dose is from about 5 wt % to about 50 wt % of the active agent loaded into the active agent matrix.

In some examples, the effective dose is an amount from about 0.01 mg to about 100 mg of the active agent.

In some examples, the device can further include the active agent composition.

In some examples, the active agent is a member selected from the group consisting of: a steroid, an antimicrobial agent, an immunosuppressive agent, a non-steroidal anti-inflammatory agent, an anti-angiogenic agent, a vasoconstrictive agent, an antihistamine, a glaucoma agent, and combinations thereof.

In some examples, the active agent is dexamethasone sodium phosphate (DSP).

In some examples, the active agent is present in the active agent composition in an amount from about 0.005 w/v % to about 25 w/v %.

In some examples, the active agent composition has a pH of from about 5 to about 8.

In some examples, the active agent composition has a tonicity of from about 250 mOsm/kg to about 750 mOsm/kg.

In some examples, the active agent composition is substantially particulate matter free.

In some examples, the device is sterile.

In some examples, the device does not include an electrode.

In some examples, the device further includes a loading base shaped to removably mate with the housing and facilitate loading of the active agent composition into the active agent matrix.

In some examples, a method of manufacturing a non-invasive ocular drug delivery device can include preparing a housing that is adapted to couple to an eye of a subject, forming an active agent matrix from an electrospun material, said active agent matrix having a combination of a density, a thickness, and a ocular surface area configured to hold and retain an active agent composition prior to application of the device to the eye, and deliver an effective dose of an active agent within 30 minutes of application of the device to the eye, and coupling the active agent matrix to the housing to form the non-invasive ocular drug delivery device.

In some examples, the housing is prepared as a monolithic unit.

In some examples, the monolithic unit is formed of a molded elastomeric material.

In some examples, the elastomeric material is a member selected from the group consisting of: ethylene propylene diene monomer (EPDM), a fluoroelastomer, an acrylonitrile-butadiene rubber, a silicone, and combinations thereof.

In some examples, the electrospun material is a hydrophilic material.

In some examples, the electrospun material is a member selected from the group consisting of: polyamides, polyurethanes, polycarbonates, polyvinyl alcohols, polylactic acids, polyglycolic acids, polyethylene-co-vinyl acetate, polyethylene oxide, polystyrene, collagen, polyvinyl pyrrolidone, polyethylene, polypropylene, and combinations thereof.

In some examples, the active agent matrix is coupled to the housing with an adhesive.

In some examples, the method further includes loading the active agent composition into the active agent matrix.

In some examples, the method further includes terminally sterilizing the non-invasive active agent delivery device.

In some examples, the method further includes aseptically sterilizing the active agent composition and the non-invasive active agent delivery device.

In some examples, a method of treating a subject with an ocular condition can include coupling a device as described herein to an eye of a subject and passively administering an active agent from the device to an eye of the subject during a continuous dosing period to provide a threshold dose of the active agent to the eye within 30 minutes of coupling the device to the eye.

In some examples, the ocular condition includes uveitis, age-related macular degeneration (AMID), macular edema, a cataract, diabetic retinopathy, glaucoma, dry eye, post-operation inflammation, eye infection, allergic conjunctivitis, presbyopia, corneal wound healing, ocular pain, or combinations thereof.

In some examples, coupling comprises inducing negative pressure between the device and the eye of the subject.

In some examples, the active agent is a member selected from the group consisting of: a steroid, an antimicrobial agent, an immunosuppressive agent, a non-steroidal anti-inflammatory agent, an anti-angiogenic agent, a vasoconstrictive agent, an antihistamine, a prostaglandin, a beta-blocker, an alpha-adrenergic agonist, a carbonic anhydrase inhibitor, an anesthetic, an analgesic, and combinations thereof.

In some examples, the threshold dose is from about 5 wt % to about 50 wt % of the active agent loaded into the active agent matrix.

In some examples, the threshold dose is an amount from about 0.01 mg to about 100 mg of the active agent.

In some examples, the threshold dose is passively delivered to the eye within 5 minutes of coupling the device to the eye.

In some examples, the threshold dose is sufficient to deliver the active agent to the posterior segment of the eye.

In some examples, the posterior segment includes the retina.

In some examples, the method further includes terminating passive administration by removing the device from the eye.

In some examples, passively administering comprising administering the threshold dose to the eye via the sclera of the eye while minimizing administration via the cornea.

It is noted that when discussing the non-invasive ocular drug delivery device, the method of manufacturing a non-invasive ocular drug delivery device, and the method of treating an ocular condition in a subject, each of these discussions can be considered applicable to each of these examples, whether or not they are explicitly discussed in the context of that example. Thus, for example, in discussing details about the device per se, such discussion also refers to the various methods described herein, and vice versa.

### EXAMPLES

### Example 1—Manufacture of a Non-Invasive Ocular Drug Delivery Device

A housing for the ocular delivery device was prepared as a monolithic unit by molding a medical grade silicone material. The housing included a corneal dome, corneal seal, and vacuum bulb as a single monolithic unit. The housing had the general appearance of that illustrated in FIGS. 3a-3d.

An active agent matrix was formed by electrospinning a hydrophilic polyether-based thermoplastic polyurethane material to form polymer fibers. The polymer fibers were used to form a non-woven pad having a dry thickness of about 0.4 mm, a dry density of about 0.25 g/cc, and a surface area of about 150 mm². Pads were manufactured to have a crescent shape so that two pads could be positioned adjacent one another about the corneal seal to deliver active agent to the sclera without contacting the cornea. The individual segments of the active agent matrix were coupled to the housing using a medical grade silicone adhesive.

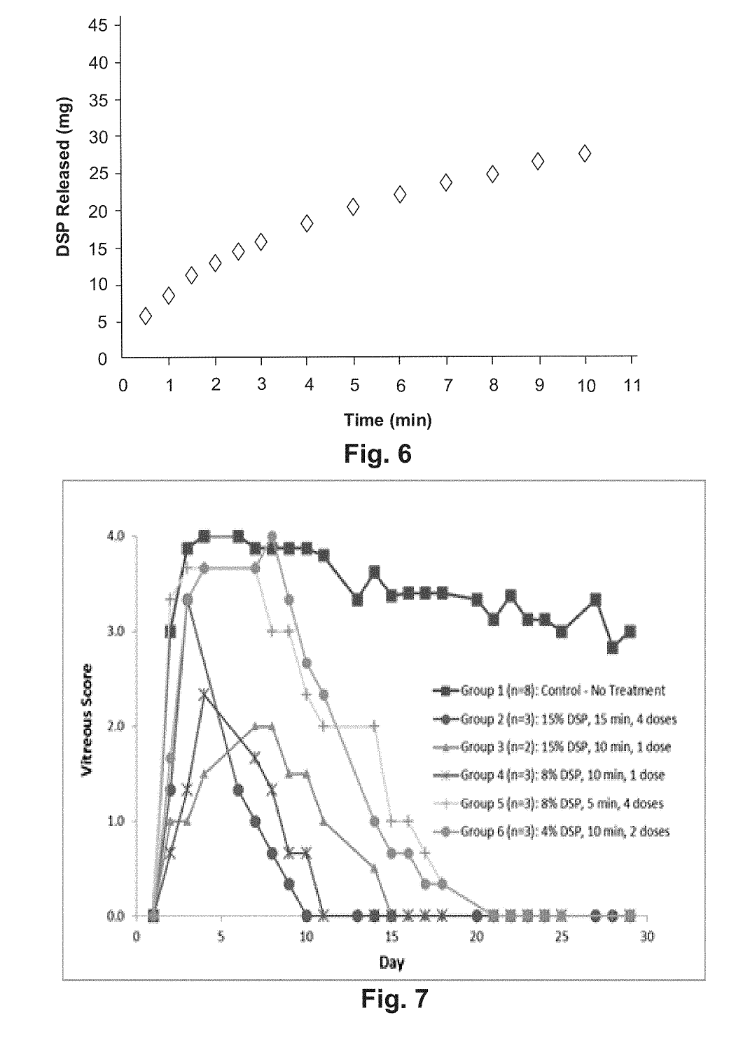

The active agent matrix was loaded with an aqueous based 15 w/v % dexamethasone sodium phosphate solution (DSP). The active agent matrix was able to passively absorb greater than 240 μL of the solution within 2 minutes. The active agent matrix did not leak, drip, or otherwise prematurely release the active agent composition. FIG. 6 illustrates an in-vitro drug release profile provided by this exemplary device.

### Example 2—Treatment of Experimental Uveitis in Rabbits

Dexamethasone sodium phosphate (DSP) USP grade was obtained from Letco Products (Decatur, Ala.). The concentrations of DSP solution were 4.0%, 8.0%, and 15.0% w/v. All DSP formulations contained 0.01% w/v of EDTA (Sigma-Aldrich, St. Louis, Mo.) with pH adjusted to 7.0 with 1M hydrochloric acid (LabChem, Zelienople, Pa.) and were freshly prepared in doubly deionized water on the day of dosing using an aseptic technique. The applicator for use in rabbit studies was fabricated from medical grade silicone rubber, which incorporated a customized active agent matrix (3-5 mm wide). Young adult New Zealand White rabbits (both male and female), each weighing 3-4 kg, were obtained from Western Oregon Rabbit Co. (Philomath, Oreg.). This study complied with the ARVO Statement for the use of Animals in Ophthalmic and Vision Research and was approved by The University of Utah Institutional Animal Care and Use Committee (Salt Lake City, Utah). All animals were acclimated and observed for health issues for at least 2 weeks before being used in the study. Freund's complete adjuvant (FCA) and Mycobacterium tuberculosis H37Ra antigen were purchased from Difco Laboratories, Inc. (Detroit, Mich.), ketamine hydrochloride injectable USP (100 mg/mL), and sodium chloride 0.9% USP were from Hospira, Inc. (Lake Forest, Ill.). Proparacaine hydrochloride ophthalmic solution and gentamicin sulfate ophthalmic solution were from Bausch & Lomb (Tampa, Fla.). Cyclopentolate hydrochloride ophthalmic solution was from Alcon Laboratories (Fort Worth, Tex.). The binocular indirect ophthalmoscope used was the Keeler All Pupil II from Keeler Instruments (Broomall, Pa.) and it was complemented with the double aspheric lens 20 D/50 mm for posterior chamber examination from Volk Optical, Inc. (Mentor, Ohio).

Twenty-three animals were randomly assigned into 6 groups according to Table 1 after uveitis induction of the right eye. Left eyes were not induced with uveitis to provide some vision in the animals throughout the study. The DSP treatment was on the affected eye (right eye). The first dose occurred ˜30 minutes after the uveitis induction on Day 1. Ocular examinations and clinical observation were performed during the weekday before and after each dosing. Following the final observations on Day 29, animals were anesthetized with a 2.5 mL intramuscular injection containing 5 mg ketamine and 30 mg xylazine per mL. Depth of anesthesia was confirmed by absence of corneal blink reflex or toe pinch response to ensure humane euthanasia. The animal was then sacrificed by an intracardiac injection of 2 mL of saturated KCl with a 3 mL syringe and 18 GA×1″ needle. The eyes were collected and processed for histological evaluation. The severity of the uveitic conditions limited the number of rabbits per group to 3 in the first part of the study. With the successful experience of the first part of the study, the same number of animals per group was kept for the rest of the study. The study was conducted in 3 parts, and each time a control group was evaluated with the treatment group(s). Then, the results were pooled for analysis.

TABLE 1
Study Design

| | | DSP | Application | |
|---|---|---|---|---|
| | Number of | Concentration | Time | |
| Group | Animals | (w/v %) | (Minutes) | Day of Dosing |
| 1 | 8 | No Treatment | n/a | n/a |
| 2 | 3 | 15 | 15 | 1, 8, 15, 22 |
| 3 | 3 | 15 | 10 | 1 |
| 4 | 3 | 8 | 10 | 1 |
| 5 | 3 | 8 | 5 | 1, 8, 15, 22 |
| 6 | 3 | 4 | 10 | 1, 8 |

For uveitis induction, rabbits were preimmunized by subcutaneous injections of 0.5 mL FCA H37Ra, a suspension of Mycobacterium tuberculosis H37Ra antigen in FCA. The Freund's Complete Adjuvant H37Ra containing 20 mg/mL of antigen was prepared by mixing dried M. tuberculosis H37Ra antigen with the FCA. The preimmunized injections were in the dorsal area of the animal's neck and occurred at 19 and 12 days before induction of uveitis. Then uveitis was induced on Day 1 by 100 mL IVT injection of a suspension containing 33 mg of the M. tuberculosis H37Ra antigen in sterile balanced salt solution on the right eye using Hamilton syringe with a 30 Ga×½ needle. No uveitis induction was performed on the left eye. Although a second IVT induction was planned on Day 15, it was not given due to the severity of inflammation in the control group eyes (Group 1). Rabbits were anesthetized with a 2.5 mL intramuscular injection containing 5 mg ketamine and 30 mg xylazine per mL. One drop each of proparacaine and gentamicin was administered to the eye before the IVT injection. The IVT injection entered through the limbus in the superior portion of the sclera and administered approximately in the middle of the vitreous.

Each rabbit was placed in a rabbit restrainer to limit movement during the DSP administration. One drop of sterile proparacaine hydrochloride ophthalmic solution, a local anesthetic, was given to the right eye of each rabbit ˜5 min before dose administration. DSP solution (250 μL) was loaded into the applicator using an Eppendorf pipettor. The drug solution saturated the carrier matrix uniformly within a minute. Then, the applicator containing the drug formulation was gently applied to the scleral surface of the right eye of each rabbit. The position of the applicator was checked to ensure that the drug matrix was in immediate contact with the white scleral part of the eye, but not the cornea. Digital laboratory timers were used for accurate application times (treatment duration) of 5, 10, or 15 min. After the given treatment duration, the applicator was carefully removed from the eye.

Body weights of the animals were taken upon arrival, immediately after EAU induction, and before sacrifice. All eyes of the animals (both left and right eyes) were examined by indirect ophthalmoscopy to evaluate respective effects on the cornea, conjunctiva, anterior chamber (AC), vitreous, posterior chamber, and sclera. One to 2 drops each of phenylephrine hydrochloride ophthalmic solution and cyclopentolate hydrochloride ophthalmic solution was used as a mydriatic. Observations pertaining to conjunctival injection, chemosis, discharge, and clarity of anterior and posterior segment of the eye were made, scored, and recorded. An average of all scores over the course of study was calculated for comparison. A modified McDonald-Shadduck scale was used for grading inflammation.

The enucleated eyes were stored in Davidson's solution (i.e. 34.7% deionized water, 11.1% glacial acetic acid, 32.0% ethanol, and 22.2% formalin) for 24 hrs, and then transferred to plastic conical tubes containing 20 mL of 70% ethanol in water. The eyes were sent for histopathological processing and evaluation at Colorado Histo-Prep (Fort Collins, Colo.). A central cut of the eye globe was taken as well as 2 cuts on either side of the central cut (calottes) at trim. For each eye, the central cut was placed into one cassette and the 2 calottes were placed together into a separate cassette. The tissues were processed, embedded in paraffin wax, sectioned by microtomy, and stained. Histopathology of the tissues was conducted on slides stained with hematoxylin and eosin. The pathologist who evaluated the tissues had no prior knowledge of the specific pharmacologic activity or formulation of the test articles. Standardized toxicologic pathology criteria and nomenclature for the rabbit were used to categorize microscopic tissue changes. For anterior section, the conjunctiva, cornea, AC, trabecular meshwork, iris, and ciliary body were evaluated and scored from 0 (normal) to 4 (marked) for signs of inflammation, including edema/congestion of the conjunctiva, ciliary body, cornea, inflammatory cell infiltration in the conjunctiva, cornea, AC, trabecular meshwork, iris, ciliary body, and neovascularization on the cornea. Scores from each tissue were combined to give a total inflammatory score of anterior section (maximum score=40). For posterior section, the vitreous, choroid, and retina were also scored from 0 (normal) to 4 (marked) for signs of inflammatory cell infiltration.

All scores are reported as mean−standard deviation (unless otherwise indicated). The differences in mean score between the control group and each DSP treatment group were evaluated by the Wilcoxon rank-sum test. This included vitreous score, AC score, and conjunctiva injection score from clinical observation, and inflammatory score and inflammatory cell infiltration score from histopathological examination. Differences were considered significant at P<0.05.

All right eyes showed signs of inflammation within a day after the induction. Left eyes showed no signs of inflammation through the end of the study. One rabbit in Group 3 died due to an unknown cause during the preimmunization period and before the initiation of DSP dosing. Inflammation occurred more significantly in the posterior chamber than in the AC. All treatment regimens reduced the signs of uveitis. However, the most prominent finding from ophthalmic examination in assessing the severity of uveitis is the vitreous opacity (FIG. 7). The observations from each section of the eye are as follows:

Vitreous.

All animals in the control group (Group 1) reached a severe uveitic state (i.e. scores of 3 or 4 for the vitreous), which remained on average above a score of 3 throughout the 28 days of study. Vitreous opacity increased steadily for the first 4 days after initiation of uveitis in all 5 groups. The opacity in Group 1 (control) increased the most. Scores for Group 1 animals decreased slightly around Day 13, but remained on average above a score of 3 throughout the experiment. By Day 4, Groups 2, 3, and 4 had reached the highest scores they would attain and began to decrease steadily thereafter. Group 5 scores began a steady decrease on Day 8, while those for Group 6 began to decrease on Day 10. There were clear decreases in vitreous opacity scores in all treatment groups, while the control group scores remained high. Group 2 animals showed a steady decrease in vitreous opacity scores until reaching zero on Day 10 and remaining at zero throughout the remainder of the study. Group 3 (15% DSP, 10 min, 1 dose) reached zero on Day 15, Group 5 (8% DSP, 10 min, 4 doses) on Day 11, and Groups 4 (8% DSP, 5 min, 1 dose) and 6 (4% DSP, 10 min, 2 doses) reached 0 on Days 21 and 22, respectively. Averaged vitreous scores over the course of study are presented in Table 3. Over the course of study, the average score of vitreous for the control group was 3.3-1.1 and all the DSP treatment groups (Groups 2-6) were statistically significantly lower than the control.

TABLE 3
Inflammation Scores from Clinical Observations
using Indirect Ophthalmoscope

| | Inflammation Score | | |
|---|---|---|---|
| Treatment | Conjunctival | Anterior | |
| Regimen | Injection | Chamber | Vitreous |
| Group 1 | 0.9 ± 0.8 | 0.5 ± 0.5 | 3.3 ± 1.1 |
| Group 2 | 0.5 ± 0.4 | 0.1 ± 0.2 | 0.4 ± 1.0 |
| Group 3 | 0.3 ± 0.4 | 0.1 ± 0.2 | 0.6 ± 1.2 |
| Group 4 | 0.5 ± 0.5 | 0.3 ± 0.4 | 0.4 ± 0.8 |
| Group 5 | 0.9 ± 0.8 | 0.5 ± 0.5 | 1.4 ± 1.7 |
| | (P = 0.3) | (P = 0.6) | |
| Group 6 | 0.5 ± 0.5 | 0.4 ± 0.6 | 1.4 ± 1.6 |
| | | (P = 0.1) | |

Anterior Chamber.

No hypopyon, synechia, or flare was noted in this study. Some fibrin formation in the AC was observed in all groups with slightly different degrees. The signs of inflammation in the AC were not drastic even with the control group. Average AC scores over the course of study was less than 1.0 for all groups (Table 3). The trends of the AC scores were similar for Group 1, Group 5, and Group 6. The average daily score of Group 5 was equal to that of the control group. Group 6 also had fibrin present throughout the study with an average daily score slightly lower than the controls, but not statistically significant. Group 4 (8%, 10 min, single dose) displayed a low fibrin score over the course of the study with an average of 0.3, which is significantly lower than the average of 0.5 for the control group (Group 1). Group 2 (15%, 15 min, 4 weekly doses) and Group 3 (15%, 10 min, single dose) reached an AC score of 0 within about 1 week after the first treatment. Both groups showed the averaged AC score of 0.1, which is significantly lower than the control group.

Conjunctival Injection.

Mild to moderate conjunctival injection was present in all animals and was observed throughout the study. Averaged group scores over the course of treatment are presented in Table 3. All treatment groups except Group 5 showed slightly lower average conjunctiva scores over the course of study than the control group (Group 1). The average conjunctiva scores of Group 5 were equal to the control group. There were day to day variations as well as an overall downward trend over the entire experiment in all groups (i.e. the average score ranged from 0 to 3 in the first 2 weeks and from 0 to 1 in the last 2 weeks). In Group 1, conjunctival injection declined slowly over the course of the experiment, but was still present until the end. Some irritation from placement of the DSP was observed in the DSP treatment groups. In Groups 2, 5, and 6 (multiple doses), slight increases were observed after each application followed by improvement until the next application. Conjunctival injection scores in Groups 3 and 4 (single dose) declined after Day 3, were minimal after about 10 days, and completely resolved by Day 22.

Chemosis.

Mild chemosis was found in all groups. Overall chemosis was minor, with no group having an average chemosis score greater than 1 at any point. In Group 1 animals (controls), chemosis decreased slowly, although with variation, throughout the study. Chemosis increased slightly after DSP treatment, a trend similar to that seen with conjunctival injection. Groups 2 and 5 showed mild chemosis immediately after each dosing, but resolving to 0 generally within a day. Groups 4 and 6 showed some variations in chemosis scores and reached 0 after Day 11, with Group 6 showing a slight reoccurrence on Days 16 through 18. Neither Group 3 rabbits displayed any significant chemosis.

Conjunctival Discharge.

Discharge was noted in all groups in a random manner. Discharge never exceeded a score of 1. There was an undistinguishable trend between the treatment regimens and the control.

Cornea.

A low grade of cornea cloudiness, mostly with scores of <1, was found in some rabbits in all groups (untreated control group and treatment groups). The corneal haze observed in all rabbits faded with time. Overall, the incidence and severity of corneal haze in treatment groups appeared to be lower than the control group.

Body Weight.

Group 1 animals (controls) maintained their average body weight throughout the study. Group 2, with the highest dosing of DSP (4 weekly doses of 15% for 15 min), had an average loss of body weight of 0.3 kg, or about 8%. There were no significant weight changes in any of the other treatment groups.

Histopathology of uveitis eyes—The eyes were collected at the end of the study on Day 29 for histopathology evaluation. The average inflammation scores for both anterior and posterior sections of the eyes graded by a veterinarian pathologist are presented in Table 4.

TABLE 4
Inflammation Scores and Inflammatory Cell Infiltration
Score from Histopathology Examination

| | Total Inflammatory | Inflammatory Cell Infiltration Score | |
|---|---|---|---|
| Treatment | Score of Anterior | Anterior | Posterior |
| Regimen | Section | Section | Section |
| Group 1 | 4.4 ± 2.6 | 0.7 ± 1.0 | 2.9 ± 1.2 |
| Group 2 | 0.2 ± 0.4 | 0.0 ± 0.2 | 0.1 ± 0.3 |
| Group 3 | 1.0 ± 1.1 | 0.2 ± 0.4 | 1.8 ± 1.5 |
| Group 4 | 1.8 ± 0.7 | 0.3 ± 0.7 | 1.2 ± 0.9 |
| Group 5 | 1.4 ± 1.7 | 0.2 ± 0.8 | 1.9 ± 1.6 |
| Group 6 | 1.9 ± 1.1 | 0.3 ± 0.7 | 2.9 ± 1.0 |
| | | | (P = 0.7) |

Anterior Section.

No edema or congestion of conjunctiva, ciliary body, or cornea was observed in all groups. No neovascularization on the cornea was found in this study. The total inflammatory score of anterior section was 4.4 on average for the untreated eye, whereas the DSP treatment groups were significantly lower. The efficacy of DSP treatment in the anterior section appears to be related to DSP concentrations. Groups 2 and 3, where the DSP concentration was 15%, the averaged total inflammatory scores were 0.2 and 1.0, respectively; Groups 4 and 5, where the DSP concentration was 8%, the total scores were 1.8 and 1.4, respectively; and Group 6, where the DSP concentration was the lowest at 4%, the total score was the highest among treatment groups at 1.9. Similarly, the inflammatory cell infiltrations into the anterior section of the eye were less in all DSP treatment groups compared to the control. This was reflected by the lower of inflammatory cell infiltration scores of the treatment groups compared to the control group. However, there was no obvious efficacy—concentration relationship among the treatment groups. All animals in Group 1 (untreated) had inflammatory cell infiltrations to the conjunctiva, cornea, AC, trabecular meshwork, iris, and/or ciliary body with the average inflammatory cell infiltration score of 0.7 for the whole anterior section. In contrast, the average inflammatory cell infiltration score of Group 2 (15% DSP, 15 min, 4 doses) was 0.0. No cell infiltrations in the conjunctiva, AC, trabecular meshwork, iris, or ciliary body were found in this group. For Group 3 (15% DSP, 15 min, 1 dose), Group 4 (8% DSP, 10 min, 1 dose), Group 5 (8% DSP, 5 min, 4 doses), and Group 6 (4% DSP, 10 min, 2 doses), few inflammatory cell infiltrations were found in ciliary body, conjunctiva, and/or cornea tissues, but not in the other anterior tissues (i.e., AC, trabecular meshwork, and iris) with the average inflammatory cell infiltration scores of 0.2, 0.3, 0.2, and 0.3, respectively.

Posterior Section.

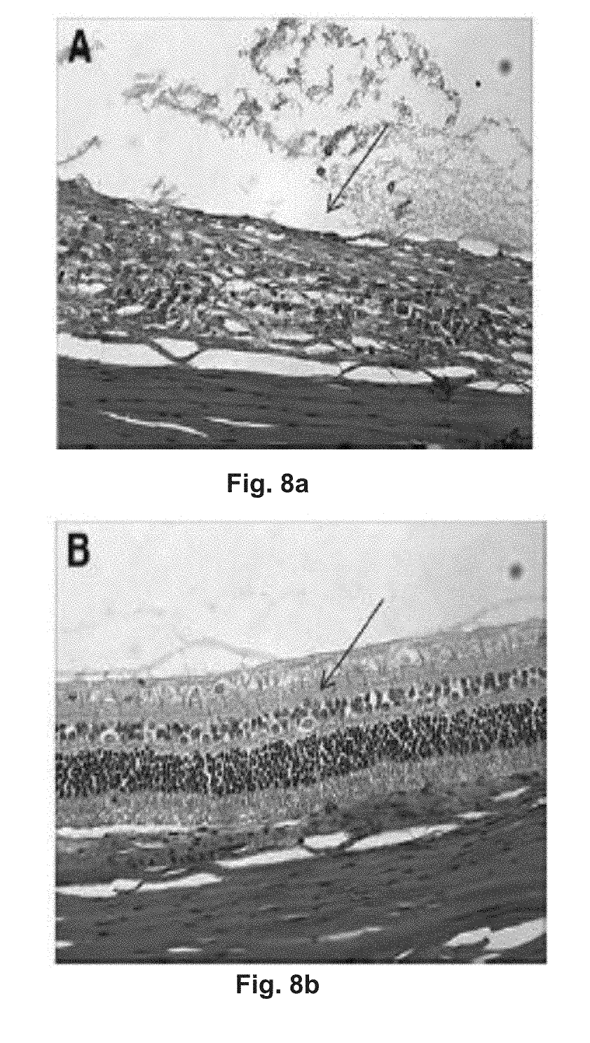

The overall inflammatory cell infiltration scores of the posterior section calculated from the respective individual vitreous, choroid, and retina scores are summarized in Table 4. The results show that all DSP treatment groups, except the lowest dosing group (Group 6), were less inflamed in the posterior section than the controls (Group 1). The untreated animals showed moderate to severe inflammation in respective vitreous, choroid, and retina tissues with the average inflammatory cell infiltration score of 2.9. This indicates that intermediate and posterior uveitis were persistent in the control group for 29 days, consistent with the clinical observations. Group 2 animals had almost no pathological signs of uveitis present, with the average inflammatory cell infiltration score of 0.1. This supports that such eyes made a full recovery from induced intermediate and posterior uveitis. The differences in the photoreceptor layer appearance between the untreated eye (Group 1) and the eye from the highest dose regimen (Group 2) can be seen in FIGS. 8a and 8b. The posterior tissues of the treated eye appeared to be healthy with minimal inflammation, where it appeared to be completely impaired in the untreated eye. Histopathology of Group 3 (15% DSP, 10 min, 1 dose), Group 4 (8% DSP, 10 min, 1 dose), and Group 5 (8% DSP, 5 min, 4 doses) showed minimal to mild inflammation with the average infiltration scores of 1.8, 1.2, and 1.9, respectively. All animals in the lowest dosing group (Group 6) had posterior section inflammation nearly identical to the control group.

### Example 3—Ocular Drug Distribution and Safety of Non-Invasive Ocular Drug Delivery System

Dexamethasone sodium phosphate (DSP) USP grade was supplied from Letco Products (Decatur, Ala.). The concentrations of DSP solution were 4.0%, 8.0%, 15.0%, and 25.0% w/v. All DSP solutions containing 0.01% w/v of EDTA (Sigma-Aldrich, St. Louis, Mo.) with the pH adjusted to 7.0 using 1.0 M hydrochloric acid (LabChem, Zelienople, Pa.) were freshly prepared in double deionized water on the day of dosing using an aseptic technique. The non-invasive ocular drug delivery device for use in this study was fabricated from medical grade silicone rubber and a proprietary sponge material. Ketamine hydrochloride injectable USP (100 mg/mL) and sodium chloride 0.9% USP were from Hospira, Inc. (Lake Forest, Ill.); proparacaine hydrochloride ophthalmic solution was from Bausch & Lomb (Tampa, Fla.); cyclopentolate hydrochloride ophthalmic solution was from Alcon Laboratories (Fort Worth, Tex.); xyrazine and potassium chloride (KCl) were from Sigma-Aldrich (St. Louis, Mo.). Syringes and needles were from Becton, Dickinson and Company (Franklin Lakes, N.J.). The binocular indirect ophthalmoscope used was the Keeler All Pupil II from Keeler Instruments (Broomall, Pa.) and it was complemented with the double aspheric lens 20 D/50 mm for posterior chamber examination from Volk Optical Inc (Mentor, Ohio). Young adult New Zealand White rabbits each weighing 3-4 kg were obtained from Western Oregon Rabbit Co. (Philomath, Oreg.). This study complied with the ARVO Statement for the use of Animals in Ophthalmic and Vision Research and was approved by The University of Utah Institutional Animal Care and Use Committee (Salt Lake City, Utah). All animals were acclimated and observed for health issues for at least two weeks prior to being used in the study.

Sixty animals were randomly assigned into twenty groups of three (n=3) for three main studies: ocular drug distribution, ocular toxicity, and toxicokinetics.

For the ocular drug distribution study, there were a total of twelve groups. The test parameters included four DSP concentrations (i.e., 4%, 8%, 15%, and 25% w/v) and three application times (i.e., 5, 10, and 20 minutes). Each group received a single DSP treatment via the non-invasive ocular drug delivery device at a pre-specified concentration and application time on both eyes concurrently (within 10-20 seconds apart). The rabbits were sacrificed immediately after dosing (generally within 5 minutes). The eyes were then enucleated and analyzed for DSP and DEX using HPLC. A total of 6 eyes were used for averaging the amount of the drug in each group. The rationale for this study was to answer whether or not a single application of the non-invasive ocular drug delivery device can deliver a meaningful amount of DSP into the deeper eye tissues. Since there is no established minimum effective concentration of DEX or DSP in ocular tissues, the target concentration of DSP in each eye tissue (immediately after the application) that is considered meaningful was arbitrarily set at 1 μg/g. This was based on the fact that 1 μg/mL DEX was the quantification limit of the HPLC assay in this study. This number can very well be on the high side as even a concentration of DEX at 10⁻⁷M (˜40 ng/mL) can inhibit prostaglandin release from rabbit coronary microvessel endothelium.

For the ocular toxicity study, there were four groups. The longest application time of interest, 20 minutes, was selected for testing safety and tolerability of the four DSP concentrations. Each rabbit received a weekly DSP administration via application of the non-invasive ocular drug delivery device (i.e., 4%, 8%, 15%, or 25% DSP concentrations) for 20 minutes in one eye (right eye) leaving the other (left eye) as an untreated control. The total exposure was 12 doses over the period of 12 weeks. Clinical observations were performed on weekdays, and before and after each dosing. Following the final observations (i.e., one week after the last dose), the rabbits were sacrificed and the eyes were processed for histological evaluation.

For the toxicokinetic study, there were four groups of rabbit. Each group received a single dose of 5 or 20 minute application of 4% or 15% of DSP in one eye. Blood was collected and processed for plasma at predose, 5, 30, 60, 120, 240, and 360 minutes, and 24, 48, 72, 96, and 168 hours after administration. Plasma concentration analysis for DEX and DSP was performed using LC-MS.

At the termination point in all three studies, the animals received a 2.5 mL intramuscular injection containing 5 mg ketamine and 30 mg xylazine per mL as general anesthetic. For each animal, the depth of anesthesia was confirmed by absence of corneal blink reflex or toe pinch response to ensure humane euthanasia. The animal was then sacrificed by an intracardiac injection of 2 mL of saturated KCl with a 3 mL syringe and 18 GA×1″ needle. The eyes were collected and processed for drug analysis or histological evaluation.

Each rabbit was placed in a rabbit restrainer to limit movement during administration of DSP via the non-invasive ocular drug delivery device. One drop of sterile proparacaine hydrochloride (a local anesthetic) was put on the eye (to be treated) 5 minutes before dose administration. DSP solution (250 μl) was loaded onto the annular active agent matrix of the non-invasive ocular drug delivery device using an Eppendorf pipettor. Then, the non-invasive ocular drug delivery device containing the DSP solution was gently applied to the scleral surface of the eye of each rabbit. The position of the device was checked to ensure that the active agent matrix was in immediate contact with the white part of the eye but not the cornea. Digital timers were used for accurate application times (i.e., 5, 10, or 20 minutes). After the given application duration, the applicator was carefully removed from the eye.

For drug analysis, the eyes were dissected into seven tissue sections: anterior chamber, lens, retina-choroid, cornea, vitreous, conjunctiva, and sclera. The anterior chamber consists of iris, ciliary muscles, and aqueous humor. After dissection, the drug was extracted from each tissue overnight with 5 mL of the extraction solvent (60% chloroform-40% methanol). The tissue was then separated from the extraction solution by centrifuge at 3400 rpm for 10 minutes. The extraction solutions were concentrated by evaporation of the solvent in a water bath at 50° C., using nitrogen gas, and then reconstituted in 1 mL of the reconstitution solvent (95% methanol/5% 1M HCl). The amounts of total DSP and DEX in the eye tissues were then determined by HPLC analysis.

For histopathology, the enucleated eyes were stored in Davidson's solution (i.e., 34.7% deionized water, 11.1% glacial acetic acid, 32.0% ethanol, and 22.2% formalin) for 24 hours and then transferred to plastic conical tubes containing 20 mL of 70% ethanol in water. The eyes were sent for histopathological processing and evaluation at Colorado Histo-Prep (Fort Collins, Colo.).

Blood was collected at predose (−20 minutes), 5, 30, 60, 120, 240, and 360 minutes, and 24, 48, 72, and 168 hours after DSP application via the non-invasive ocular drug delivery device. Approximately 1 mL of blood was collected by direct venipuncture of the jugular vein with a 3 mL syringe and 21 GA×1″ needle. Blood was immediately transferred into anticoagulant (potassium EDTA) coated microcentrifuge tubes. Blood was then centrifuged for five minutes at 3000×G at 4° C. Plasma was immediately separated into another microcentrifuge tube then kept in −20° C. freezer for LC-MS analysis.

The amounts of DSP and DEX in the eye tissues were determined by HPLC analysis. The HPLC system used was Waters 2695 separation module equipped with Waters 2487 dual wavelength detector (Waters Corporation, Milford, Mass.) and Kinetex C18 column 2.6 μm 100×4.6 mm (Phenomenex, Torrance, Calif.). All the chemical reagents for making HPLC mobile phases were HPLC grade from Sigma-Aldrich (St. Louis, Mo.). The mobile phase was 30% by volume of acetonitrile and 0.1% by volume of trifluoroacetic acid (99%) in distilled deionized water. The HPLC method was isocratic with a 1.2 mL/min flow rate and column temperature was 30° C. The injection volume was 10 μL. A single UV wavelength mode was set at λ=240 nm. Retention times for DSP and DEX were 4.2 and 6.9 min, respectively. The DSP and DEX standard curves of 0.0005 to 0.5 mg/mL (i.e., concentration vs. absorbance) were generated. The lower limit of quantification of this method was 0.001 mg/mL.

All of the plasma analyses for DSP and DEX were performed at Tandem Labs (Salt Lake City, Utah) using LCMS. Briefly, the samples were assayed by Shimadzu SCL-10A controller with LC-10AD pump. The mobile phase was 50% by volume of 10 mM ammonium acetate and 50% by volume of methanol. The HPLC column was a)(Bridge Phenyl column, 5 μm, 50×2.0 mm. An isocratic elution was applied at 0.500 mL/min flow rate and column temperature was 30° C. An API 5000 (Applied Biosystem/Sciex) mass detector with an electrospray interface in positive mode (source temperature set at 400° C.) was used to detect the MS/MS transition m/z 393 to m/z 373.4 for DEX and m/z 473 to m/z 435 for DSP. The injection volume was 10 μL. The retention times for DSP and DEX were 1.2 and 2.5 min, respectively. DSP and Dex standard curves of 0.2 to 200 ng/mL were generated. The limit of quantitation (LOQ) of this method was 1 ng/mL.

Toxicokinetic data analysis was based on standard noncompartmental pharmacokinetic methods. Plasma concentration of DSP equivalent was used in the analysis to express systemic exposure of DSP and DEX as a single entity. The DSP equivalent was calculated by converting DEX to DSP using 392.5 g of DEX equivalent to 516.4 g of DSP. The maximum observed plasma concentration (Cmax) was determined by visual estimation from the data plot. Area under the plasma concentration vs. time curve from 0 to the time of the last measurable concentration (AUC) was calculated by the linear trapezoidal method. Elimination half-life (t_{1/4}) was calculated as ln(2)/ke, where ke is the elimination rate constant determined by linear regression of the last three analytically measured points on the plasma concentration vs. time curve.

Body weights of the animal were taken upon arrival, and then monthly. All animals (both left and right eyes) were examined by indirect ophthalmoscopy of the cornea, conjunctiva, anterior chamber, vitreous, posterior chamber, and sclera. One to two drops each of phenylephrine hydrochloride and cyclopentolate hydrochloride were used as mydriatics. Observations on the anterior and posterior segments of the eye were made, graded, and recorded. A modified McDonald-Shadduck scale was used for grading eye irritation and ocular toxicity.

The histopathological processing and evaluation were conducted at Colorado Histo-Prep (Fort Collins, Colo.). Briefly, a central cut of the eye globe was taken, as well as two cuts on either side of the central cut (calottes) at trim. For each eye, the central cut was placed into one cassette, and the two calottes were placed together into a separate cassette. The tissues were processed, embedded in paraffin wax, sectioned by microtome, and stained. Histopathology of the tissues was conducted on slides stained with hematoxylin and eosin. A pathologist who evaluated the tissues had no knowledge of the specific pharmacologic activity or formulation of the test articles. Standardized toxicological pathology criteria and nomenclature for the rabbit were used to categorize microscopic tissue changes.

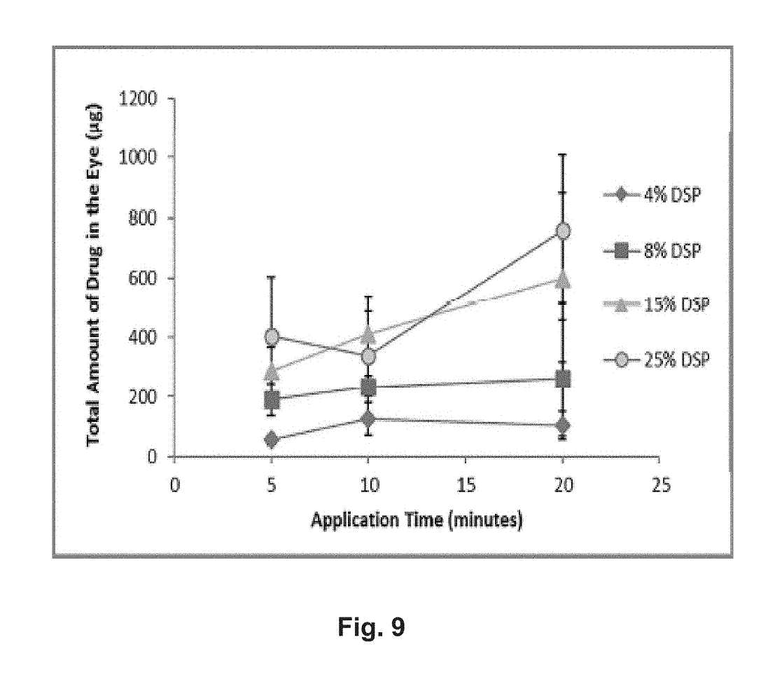

After single applications of DSP via the non-invasive ocular drug delivery device for 5, 10, or 20 minutes and for all DSP concentrations, significant amounts of DSP and some DEX were found in all the tissues. A typical rank order of DSP amounts in the eye tissue is sclera, conjunctiva, cornea, retina-choroid, anterior chamber, vitreous, and lens. The total amount of drugs in each tissue except vitreous and lens appears to be correlated well with the DSP concentration and application time of the non-invasive ocular drug delivery device. In FIG. 9, the total amount of DSP delivered by the non-invasive ocular drug delivery device was calculated by the sum of DSP and DEX in μg for a purpose of drug delivery analysis. Generally, at a given application duration (i.e., 5, 10, or 20 minutes), a higher DSP formulation concentration yielded a higher amount of DSP in the eye. Similarly, at a given concentration, a longer application duration of the non-invasive ocular drug delivery device yielded a higher amount of DSP in the eye.

The concentration of DSP in each tissue was also calculated in μg/g and summarized in Table 5 for potential efficacy evaluation of the non-invasive ocular drug delivery device. As discussed earlier, the concentration of 1 μg/g or higher in the tissue is considered as a potential therapeutic level. With exception of the lens and vitreous samples in a few cases, most of the ocular tissue concentrations of DSP are significantly higher than 1 μg/g. The typical order of concentration of DSP in ocular tissues, from high to low, was cornea >sclera >conjunctiva >retina-choroid >anterior chamber >lens >vitreous. The drug concentration in the ocular tissues (except lens and vitreous,) correlated well with both increasing DSP concentration in the non-invasive ocular drug delivery device and treatment duration.

TABLE 5
DSP-equivalent concentrations in ocular tissues (mean ± SD, μg/g).

| | | Aqueous | | | Retina- | | |
|---|---|---|---|---|---|---|---|
| Dose | Cornea | Chamber | Lens | Vitreous | Choroid | Sclera | Conjunctiva |
| 4% DSP, | 108 ± 74 | 14 ± 4 | 0 ± 0 | 0 ± 1 | 18 ± 16 | 59 ± 25 | 33 ± 29 |
| 5 min | | | | | | | |
| 4% DSP, | 216 ± 86 | 11 ± 8 | 2 ± 0 | 2 ± 1 | 59 ± 86 | 131 ± 52 | 56 ± 14 |
| 10 min | | | | | | | |
| 4% DSP, | 147 ± 75 | 12 ± 4 | 3 ± 1 | 1 ± 1 | 24 ± 7 | 154 ± 49 | 49 ± 26 |
| 20 min | | | | | | | |
| 8% DSP, | 288 ± 73 | 23 ± 3 | 13 ± 0 | 5 ± 1 | 74 ± 23 | 233 ± 53 | 84 ± 36 |
| 5 min | | | | | | | |
| 8% DSP, | 459 ± 148 | 23 ± 9 | 0 ± 0 | 2 ± 1 | 63 ± 61 | 314 ± 74 | 104 ± 33 |
| 10 min | | | | | | | |
| 8% DSP, | 567 ± 397 | 56 ± 54 | 14 ± 24 | 6 ± 8 | 54 ± 38 | 306 ± 207 | 113 ± 58 |
| 20 min | | | | | | | |
| 15% DSP, | 367 ± 118 | 18 ± 3 | 5 ± 0 | 5 ± 3 | 182 ± 176 | 328 ± 60 | 150 ± 26 |
| 5 min | | | | | | | |
| 15% DSP, | 467 ± 173 | 43 ± 11 | 17 ± 1 | 7 ± 1 | 113 ± 32 | 512 ± 54 | 222 ± 45 |
| 10 min | | | | | | | |
| 15% DSP, | 1128 ± 521 | 89 ± 42 | 13 ± 3 | 12 ± 3 | 351 ± 275 | 615 ± 336 | 287 ± 94 |
| 20 min | | | | | | | |
| 25% DSP, | 714 ± 252 | 39 ± 17 | 6 ± 1 | 6 ± 3 | 114 ± 82 | 452 ± 214 | 221 ± 126 |
| 5 min | | | | | | | |
| 25% DSP, | 512 ± 327 | 35 ± 32 | 1 ± 1 | 4 ± 4 | 60 ± 77 | 429 ± 231 | 184 ± 109 |
| 10 min | | | | | | | |
| 25% DSP, | 2225 ± 886 | 169 ± 67 | 13 ± 4 | 9 ± 4 | 207 ± 101 | 731 ± 189 | 347 ± 107 |
| 20 min | | | | | | | |

Over the course of the 12 week toxicity study entailing 12 weekly doses of DSP via the non-invasive ocular drug delivery device, ocular findings noted with the treated eyes (right eye) were conjunctival injection, discharge, and corneal haze. These ocular findings were transient and mild in nature. No abnormalities or signs of ocular toxicity were observed in untreated eyes (left eye). Details of the ocular findings are given below and a summary of the clinical observations over 12 weeks including the conjunctival injection scores, histopathological results, and body weight is presented in Table 6.

Conjunctiva: Conjunctival injection was generally observed immediately after DSP application in all groups. Resolution period of conjunctival injection correlates with DSP concentration. As the DSP concentration increased, it took longer times to resolve to the baseline. The resolution period of conjunctival injection was generally within 1-2 days for 4% and 8% DSP and up to 7 days for 15% and 25% DSP in some cases. The average conjunctival scores for every 4 weeks indicate that the degree of conjunctival injection increased with the DSP concentration and repeated applications (see Table 6). The animals treated with 4% and 8% DSP had typical conjunctival injection scores immediately after treatment of 1 or <1 through the whole study. In a rare occasion, a score of 2 was found in the 8% DSP group. The animals treated with 15% DSP had typical conjunctival injection scores immediately after treatment of <1 for the first four weeks, and then 2 at Week 8 until the end of study. The animals treated with 25% DSP had typical conjunctival injection scores immediately after treatment of <1 for the first four weeks, and then 2 or 3 at Week 8 until the end of study. Chemosis on the conjunctiva was also observed immediately after DSP administration via the non-invasive ocular drug delivery device. Although chemosis tends to increase in severity with the DSP concentration and with repeated application, the occurrence of chemosis appeared to be sporadic. Conjunctival discharge was noted occasionally but appears to be irrespective of DSP concentration and not related to infection.

TABLE 6
Clinical Observations

| | Average Conjunctival Injection Score (range) | | | | | | Body | Ocular |
|---|---|---|---|---|---|---|---|---|
| Dose | Weeks 1-4 | | Weeks 5-8 | | Weeks 9-12 | | Weight | Histopathology |
| 4% DSP, 20 min | 0.11 | (0 to <1) | 0.25 | (0 to 1) | 0.31 | (0 to 1) | NCS | NSF |
| 8% DSP, 20 min | 0.11 | (0 to <1) | 0.19 | (0 to 1) | 0.40 | (0 to 2) | NCS | NSF |
| 15% DSP, 20 min | 0.27 | (0 to 1) | 0.72 | (0 to 2) | 0.93 | (0 to 2) | 8% loss | NSF |
| 25% DSP, 20 min | 0.36 | (0 to 1) | 1.08 | (0 to 3) | 1.39 | (0 to 3) | 13% loss | NSF |

NCS = No clinically significant change.
NSF = No significant findings.

Cornea: Cornea appeared normal after each DSP administration via the non-invasive ocular drug delivery device in all rabbits except in one case with a rabbit in the 15% DSP group from Week 4 to Week 8. Corneal haze on the treated eye was immediately observed in this rabbit after the DSP administration on Week 4. The lesion covered about 40% of the corneal surface. The haze was identified as a result of an off center applicator placement. This caused the drug reservoir to be in direct contact with the cornea during the DSP administration via the non-invasive ocular drug delivery device. The corneal haze grew fainter over time and it was not visible by Week 8.

Body Weight: There were no significant weight changes in the 4% or 8% DSP treated rabbits. However, the animals in the 15% and 25% DSP groups showed trends of decreasing body weight. The consistent decline in body weights of the animals in these two groups indicate that long term exposure at these levels of DSP dosing (i.e., 15% and 25% DSP for 20 min) may have significant systemic side effects on rabbit.

Histopathology: All eyes were considered to be morphologically normal, except one treated eye in the 8% DSP group showed mild chronic inflammation at the limbus of the cornea. Besides that one eye, there were no significant findings (NSF) with any ocular tissue examined. No test article changes were identified.

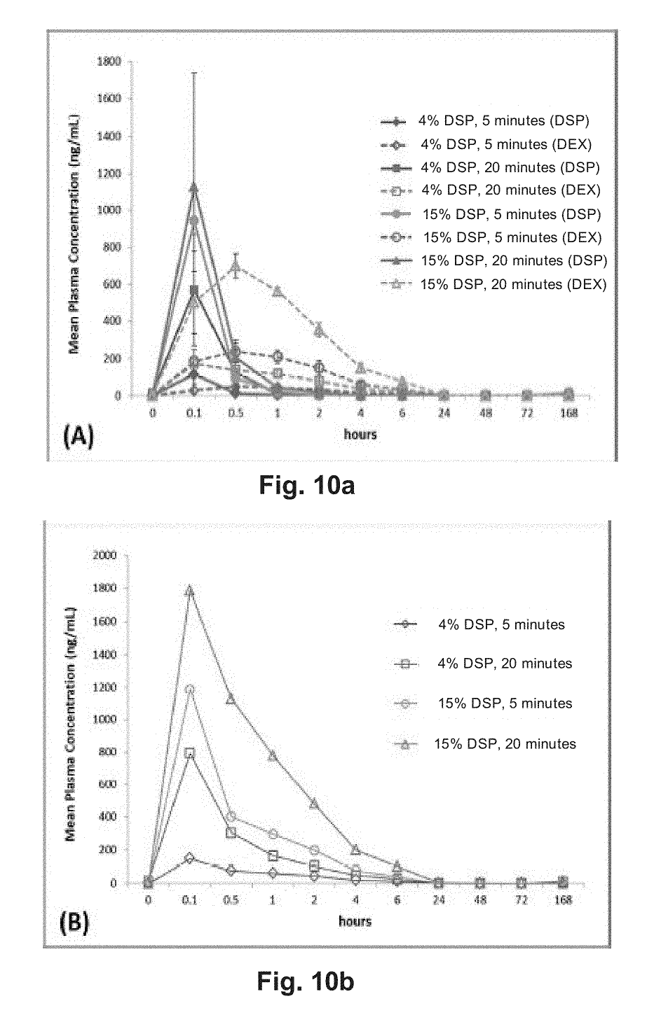

After single applications of the non-invasive drug delivery device, DSP and DEX were found in plasma for all four treatment regimens (i.e., 5 or 20 minute applications of 4% or 15% of DSP). The plasma concentrations of DSP and DEX after single applications of the non-invasive ocular drug delivery device are shown in FIG. 10a. Tmax of DSP was reached at the first blood draw (5 minutes after device application) whereas Tmax of DEX was reached later at 30 minutes. The maximum plasma concentration (Cmax) of both DSP and DEX increased with increasing DSP concentration and with longer application time. It appears that the concentration affected the systemic exposure more than the application time; the 4% DSP applied for 20 minutes yielded a lower plasma concentration than the 15% DSP applied for 5 minutes. Within 24 hours, the drug plasma concentrations of all groups were approaching or under the lowest detection limit of 1 ng/mL.

For the purpose of assessing the systemic exposure of DSP and DEX, the DSP and DEX plasma concentrations were combined and calculated as DSP equivalent. The DSP equivalent is defined as the sum of DSP and DEX in gram equivalent, with 392.5 g of DEX equivalent to 516.4 g of DSP. The pharmacokinetic profiles of DSP equivalent from all four treatment regimens are shown in FIG. 10b and the key toxicokinetic parameters are presented in Table 7, The half-life of the drug in the rabbit is approximately 2-3 hours. Cmax and AUC increased with increased concentration of DSP and increased application time.

To put the systemic DSP exposure in rabbit into human perspective, estimations of Cmax of the DSP in human were made and presented in Table 7. Cmax values in human were estimated based on Cmax data from IV injections in both rabbit and human: IV injection of 1 mg DSP yields a Cmax of 786 ng/mL in rabbit and 10.5 ng/mL in human. These results suggest that the Cmax of DSP for rabbit is approximately 75 times higher than that for human. The estimated Cmax in human of the lowest dose (4% DSP, 5 minutes) and the highest dose (15% DSP, 20 minutes) of DSP administered via the non-invasive ocular drug delivery device are 2 and 25 ng/mL, respectively.

TABLE 7
DSP-Equivalent Concentrations in Plasma

| | | | | Estimated |
|---|---|---|---|---|
| | | | | Cₘₐₓ in |
| | | | AUC | Human |
| Dose | Cₘₐₓ (ng/ml) | t_{1/2} (h) | (ng*h/ml) | (ng/ml) |
| 4% DSP, 5 min | 148 ± 71 | 3.1 ± 2.2 | 418 ± 93 | 2 ± 1 |
| 4% DSP, 20 min | 795 ± 344 | 2.3 ± 0.6 | 996 ± 144 | 11 ± 5 |
| 15% DSP, 5 min | 1188 ± 306 | 1.7 ± 0.9 | 1595 ± 418 | 16 ± 4 |
| 15% DSP, 20 min | 1844 ± 664 | 2.7 ± 0.3 | 3779 ± 472 | 25 ± 9 |

### Example 4—Aging Study for Non-Invasive Ocular Drug Delivery Device

A non-invasive ocular drug delivery device was prepared as described in Example 1. The device was sterilized using electron beam irradiation and subsequently stored at 25° C./60% RH for 24 months. At various time points the device was examined to determine uptake of 190±10 μL of 15% dexamethasone sodium phosphate (DSP) in aqueous vehicle within 120 seconds. Subsequently, the device was evaluated to determine the amount of absorbed DSP released over 10 minutes. Additionally, intentional and unintentional separation force of the device were measured at each time point. Specifically, the upper portion of the bulb was squeezed and the device was pressed lightly onto a corneal seal fixture until the bulb began to visually compress such that an annular vacuum seal was formed around the corneal seal of the device. Without dislodging the device, an open end of a separation fixture was positioned around a portion of the applicator and the other end of the separation fixture was connected to a tensile testing machine. The tensile strain rate of the tensile testing machine was set to 400 mm/min and was run until the device had completely separated from the corneal seal fixture. The peak load required to create the separation was recorded. It is noted that with the intentional separation model, the device bulb was directly secured to the tensile testing machine such that jaws of the machine compressed the bulb to expel the vacuum without dislodging the device from the corneal seal fixture. Otherwise, the test was the same. Further, a “Force at Break” test was performed at each time point. Specifically, a stainless steel razor blade was used to score away the silicone device where the drug matrix ends met to allow free access to the drug matrix material. The bulb of the device was placed in a lower jaw of the tensile testing machine and the exposed drug matrix material was placed in an upper jaw of the tensile testing machine. The test was then run until the drug matrix separated from the applicator. The peak force was recorded. Table 8 below illustrates that results of the post-sterilization device integrity study.

TABLE 8
Non-Invasive Ocular Drug Delivery Device Post-Sterilization Testing

| | Pre-Sterilization | Post-Sterilization | | | | | |
|---|---|---|---|---|---|---|---|
| Specification | 0 Month (M) | 0 M | 3 M | 6 M | 12 M | 18 M | 24 M |
| Drug | 139 ± 11 | 142 ± 9 | 143 ± 8 | 141 ± 8 | 146 ± 7 | 145 ± 6 | 146 ± 8 |
| Absorption | | | | | | | |
| (μL) | | | | | | | |
| Drug Release | 13 ± 2 | 9 ± 1 | 9 ± 2 | 10 ± 1 | 10 ± 2 | 9 ± 2 | 10 ± 1 |
| (mg) | | | | | | | |
| Force at Break | n/a | n/a | 1.36 ± 0.40 | 1.42 ± 0.32 | 1.49 ± 0.26 | 1.28 ± 0.28 | 1.06 ± 0.30 |
| (N) | | | | | | | |
| Unintentional | 1.64 ± 0.38 | 2.03 ± 0.30 | 1.51 ± 0.04 | 1.71 ± 0.33 | 2.54 ± 0.43 | 1.92 ± 0.16 | 1.77 ± 0.38 |
| Separation (N) | | | | | | | |
| Intentional | 0.32 ± 0.17 | 0.60 ± 0.28 | 0.34 ± 0.28 | 0.45 ± 0.12 | 0.59 ± 0.22 | 0.46 ± 0.14 | 0.34 ± 0.20 |
| Separation (N) | | | | | | | |

It should be understood that the above-described methods are only illustrative of some embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that variations including, may be made without departing from the principles and concepts set forth herein.

What is claimed is:

1. A non-invasive and passive ocular drug delivery device, comprising:
a housing configured to couple to an eye of a subject; and
an active agent matrix coupled to the housing, said active agent matrix comprising an electro spun material having a combination of a density, a thickness, and an ocular surface area configured to hold and retain an active agent composition prior to application of the device to the eye, and deliver an effective dose of an active agent within 30 minutes of application of the device to the eye, said active agent composition comprising the active agent.

2. The device of claim 1, wherein the active agent matrix is positioned to interface with a sclera of the eye, but not a cornea of the eye.

3. The device of claim 1, wherein the active agent matrix is coupled to the housing via an adhesive.

4. The device of claim 3, wherein the adhesive is a member selected from the group consisting of: a silicone adhesive, an epoxy adhesive, an acrylic adhesive, a polyurethane adhesive, and combinations thereof.

5. The device of claim 1, wherein the electrospun material is a hydrophobic material.

6. The device of claim 1, wherein the electrospun material is a hydrophilic material.

7. The device of claim 1, wherein the electrospun material is a polyurethane material.

8. The device of claim 1, wherein the active agent matrix is solvent-absorbable to a weight of from about 2 times to about 25 times the dry weight of the active agent matrix.

9. The device of claim 1, wherein the density of the active agent matrix is from about 0.15 grams/cubic centimeter (cc) to about 0.4 grams/cc prior to loading.

10. The device of claim 1, wherein the thickness of the active agent matrix is from about 250 μm to about 600 μm prior to loading.

11. The device of claim 1, wherein the ocular surface area of the active agent matrix is from about 50 mm² to about 300 mm².

12. The device of claim 1, wherein the active agent matrix has a loading capacity to hold and retain from about 50 μL to about 5000 μL of the active agent composition prior to application.

13. The device of claim 1, wherein the active agent matrix is configured to passively absorb at least 100 μL of the active agent composition within 10 minutes.

14. The device of claim 1, wherein the active agent matrix is configured to deliver the effective dose of the active agent within 20 minutes of application of the device to the eye.

15. The device of claim 1, wherein the effective dose is from about 1 wt % to about 50 wt % of the active agent loaded into the active agent matrix.

16. The device of claim 1, wherein the effective dose is an amount from about 0.001 mg to about 100 mg of the active agent.

17. The device of claim 1, further comprising the active agent composition.

18. The device of claim 17, wherein the active agent is a member selected from the group consisting of: a steroid, an antimicrobial agent, an immunosuppressive agent, a non-steroidal anti-inflammatory agent, an anti-angiogenic agent, a vasoconstrictive agent, an antihistamine, a glaucoma agent, an anesthetic, an analgesic, and combinations thereof.

19. The device of claim 17, wherein the active agent is dexamethasone sodium phosphate (DSP).

20. The device of claim 17, wherein the active agent is present in the active agent composition in an amount from about 0.005 w/v % to about 25 w/v %.

21. A method of treating a subject with an ocular condition, comprising:
coupling a device according to claim 1 to an eye of a subject; and
passively administering an active agent from the device to an eye of the subject during a continuous dosing period to provide a threshold dose of the active agent to the eye within 30 minutes of coupling the device to the eye.

22. The method of claim 21, wherein the ocular condition includes uveitis, age-related macular degeneration (AMD), macular edema, a cataract, diabetic retinopathy, glaucoma, dry eye, post-operation inflammation, eye infection, allergic conjunctivitis, presbyopia, corneal wound healing, ocular pain, or combinations thereof.

23. The method of claim 21, wherein coupling comprises inducing negative pressure between the device and the eye of the subject.

24. The method of claim 21, wherein the active agent is a member selected from the group consisting of: a steroid, an antimicrobial agent, an immunosuppressive agent, a non-steroidal anti-inflammatory agent, an anti-angiogenic agent, a vasoconstrictive agent, an antihistamine, a prostaglandin, a beta-blocker, an alpha-adrenergic agonist, a carbonic anhydrase inhibitor, an anesthetic, an analgesic, and combinations thereof.

25. The method of claim 21, wherein the threshold dose is from about 1 wt % to about 50 wt % of the active agent loaded into the active agent matrix.

26. The method of claim 21, wherein the threshold dose is an amount from about 0.001 mg to about 100 mg of the active agent.

27. The method of claim 21, wherein the threshold dose is passively delivered to the eye within 5 minutes of coupling the device to the eye.

28. The method of claim 21, wherein the threshold dose is sufficient to deliver the active agent to the posterior segment of the eye.

29. The method of claim 21, further comprising terminating passive administration by removing the device from the eye.

30. The method of claim 21, wherein passively administering comprising administering the threshold dose to the eye via the sclera of the eye while minimizing direct administration via the cornea.
